(19) United States
(12) Patent Application Publication
(10) Pub. No.: US 2004/0110803 A1
(43) Pub. Date: Jun. 10, 2004

(54) Methods and compositions for the use of D-malic acid to decrease serum triglyceride, cholesterol and lipoprotein levels

(76) Inventors: Hossein Dovlatabadi, Birmingham, AL (US); Ronald L. Jenkins, Birmingham, AL (US)

Correspondence Address: KING & SPALDING LLP, 191 PEACHTREE STREET, N.E., ATLANTA, GA, 30303-1763, US

(21) Appl. No.: 10/662,654

(22) Filed: Sep. 15, 2003

Related U.S. Application Data
(60) Provisional application No. 60/410,866, filed on Sep. 13, 2002.

(51) Int. Cl. A61K 31/44; A61K 31/401; A61K 31/195; A61K 31/19

(52) U.S. Cl. 514/355; 514/423; 514/557; 514/563

(57) ABSTRACT

Compositions, methods and uses are provided for treating or preventing cardiovascular disease, including by decreasing serum cholesterol, triglyceride and lipoprotein cholesterol levels in a host that include administering an effective amount of D-malic acid or its pharmaceutically acceptable salt, prodrug or pharmaceutically acceptable derivative.

This application claims priority to U.S. S. No. 60/410,866, filed on Sep. 13, 2002.

### FIELD OF THE INVENTION

This invention is in the area of compositions and methods to decrease serum triglyceride, total cholesterol, low density and very low density lipoprotein cholesterol levels using D-malic acid or a pharmaceutically acceptable salt, prodrug or active derivative thereof.

### BACKGROUND OF THE INVENTION

Hyperlipidemia is manifested in people of all ages, races, occupations, and ethnic origins and is thought to be influenced by genetics, diet, disease state, and level of daily activity. The consequences of hyperlipidemia and its sequellae on the human population are staggering, correlated to high incidence of high blood pressure, heart disease, atherosclerosis, diabetes, and cancer (Salonen, et al. 1995).

Hyperlipidemia is also believed to contribute to coronary heart disease (CHD) which remains the leading cause of death in the industrialized countries. The primary cause of CHD is atherosclerosis, a disease characterized by the deposition of lipids, including cholesterol, in the arterial vessel wall, resulting in a narrowing of the vessel passages and ultimately hardening of the vascular system.

Atherosclerosis generally begins with local injury to the arterial endothelium followed by proliferation of arterial smooth muscle cells from the medial layer to the intimal layer along with the deposition of lipid and accumulation of foam cells in the lesion. As the atherosclerotic plaque develops it progressively occludes more and more of the affected blood vessel and can eventually lead to ischemia or infarction. Because deposition of circulating lipids such as cholesterol plays a major role in the initiation and progression of atherosclerosis, it is important to identify compounds, methods and compositions to help remove cholesterol from the developing peripheral tissues, including atherosclerotic plaque.

Circulating lipoproteins serve as vehicles for the transport of water-insoluble lipids like cholesteryl esters, triglycerides and the more polar phospholipids and unesterified cholesterol in the aqueous environment of plasma (Bradely, W. A. and Gotto, A. M.: American Physiological Society, Bethesda, Md., pp 117-137 (1978)). The solubility of these lipids is achieved through physical association with proteins termed apolipoproteins, and the lipid-protein complexes are called lipoproteins (Dolphin, P. J., Can. J. Biochem. Cell. Biol. 63, 850-869 (1985)). Five distinct classes of lipoproteins have been isolated from human plasma: chylomicrons, very low-density lipoproteins (VLDL), low density lipoproteins (LDL), high-density lipoproteins (HDL) and lipoprotein (a) (LP(a)). (Alaupovic, P. (1980) In Handbook of Electrophoresis. Vol. 1, pp. 27-46; Havel, R. J., Eder, H. A.; Bragdon, J. H., J. Clin. Invest. 34, 1343 (1955)).

HDL particles are first secreted from the liver and intestine as small, discoidal particles called “pre-beta 1” HDL. HDL particles undergo a continuous interconversion in the plasma beginning with the conversion of the “nascent discoidal “pre-beta 1” HDL into spherical HDL3, through the action of plasmatic enzymes, mainly lecithin-cholesteryl acyltransferase (LCAT), that converts free cholesterol to cholesteryl ester (CE) (Glomset J. A., and Norum K. R., Advan. Lipid Res., 11, 1-65, (1973); McCall, M. R., Nichols, A. V., Morton, R. E., Blanche, P. J., Shore, V. G., Hara, S. and Forte, T. M., J. Lipid Res. 34, 37 (1993)). HDL3 acquires phospholipids (PL) and free cholesterol in the presence of other plasmatic enzymes such as lipoprotein lipase (LPL) (Patsch, J. R., Gotto, A. M., Olivercrona, T. and Eisenberg, S., Proc. Natl. Acad. Sci., 75, 4519 (1978)), and further action of LCAT helps form large CE-rich HDL which constitute the CE-rich HDL2 subpopulation (McCall, M. R., et al., J. Lipid Res. 34, 37 (1993)). Mature HDL is spherical and contains various amounts of lipids and apolipoprotein. Apolipoprotein A-I (apoAI) is the major protein component of mature HDL, and most of the cholesterol associated with HDL is esterified as cholesteryl esters. HDL is believed to play a fundamental functional role in the transport of lipids and represents a site for storage of potentially harmful lipids and apolipoproteins which if unregulated could have harmful effects including changing cellular functions, altering gene expression, and obstructing blood flow by narrowing the vessel lumen. Apolipoprotein A-I has been found to be more powerful as a marker for coronary disease than the cholesterol component of HDL (Maciejko J. J. et al., New England J. Med. 309, 385-389 (1983)). However, HDL remains an important independent predictor of atherosclerosis, and HDL is an important predictor of survival in post coronary artery bypass graft patients as a result of the 20-year experience from The Cleveland Clinic Foundation (Foody J M et al. (2000) Circulation, 102 (19 suppl 3), III90-94). Clinical surveys have confirmed that elevated HDL is favorable in preventing the development of atherosclerotic lesion and low levels of HDL together with low apoAI levels are currently considered to be the most reliable parameters in predicting the development of atherosclerosis in hyperlipidemic patients (Mingpeng S. and Zongli W., (1999) Experimental Gerontology, 34 (4); 539-48).

Existing Lipid Therapies

In recent years several promising options for treating hyperlipidemia have come available, however each with their therapeutic limitation. Nicotinic acid (niacin) has been effective in lowering LDL from 10% to 20%. The HMG CoA reductase inhibitors have been effective as a primary therapy for mild hypercholesterolemia in adults of all ages and lowers serum triglycerides by 30% and LDL cholesterol by 25% to 45%. (Jukema, et al. 1995). HMG CoA reductase inhibitors often have serious hepatic contra-indications in addition to interactions with various antibiotics and CNS toxicity.

U.S. Pat. No. 5,948,435 discloses a method of regulating cholesterol related genes and enzymes by administering lipid acceptors such as liposomes. Additionally, U.S. Pat. No. 5,746,223 discloses a method of forcing the reverse transport of cholesterol by administering liposomes.

Several known agents such as Gemfibrozil (Kashyap, A., Art. Thromb. Vasc. Biol. 16, 1052 (1996)) increase HDLc levels. Gemfibrozil is a member of an important class of drugs called fibrates that act on the liver. Fibrates are fibric acid derivatives (bezafibrate, fenofibrate, gemfibrozil and clofibrate) which profoundly lower plasma triglyceride levels and elevate HDL (Sirtori C. R., and Franceschini G., Pharmac Ther. 37, 167 (1988); Grundy S. M., and Vega G. L. Amer. J. Med. 83, 9 (1987)). The typical clinical use of fibrates is in patients with hypertriglyceridemia, low HDL and combined hyperlipidemia.

The mechanism of action of fibrates is not completely understood but involves the induction of certain apolipoproteins and enzymes involved in VLDL and HDL metabolism. For example, CETP activity is reduced by fenofibrate, gemfibrozil, phentyoin and alcohol.

Nicotinic acid (niacin), a water-soluble vitamin has a lipid lowering profile similar to fibrates and may target the liver. Niacin has been reported to increase apoAI by selectively decreasing hepatic removal of HDL apoAI, but niacin does not increase the selective hepatic uptake of cholesteryl esters (Jin, F. Y., et al., Arterioscler. Thromb. Vasc. Biol. 17, 2020 (1997)).

Diet contributes up to 40% of cholesterol that enters through the intestine and bile contributes the rest of the “exogenous” cholesterol absorbed through the intestine (Wilson M. D., and Rudel L. L. J. Lipid Res. 35, 943 (1994)). Decreasing dietary cholesterol absorption therefore is a regulatory point for cholesterol whole body homeostasis. Cholesterol absorption inhibitors lower plasma cholesterol by reducing the absorption of dietary cholesterol in the gut or by acting as bile acid sequestrants (Stedronsky, E. R., Biochim. Biophys. Acta 1210, 255 (1994)).

Cholesterol lowering agents decrease total plasma and LDL and some may increase HDL. For example, statins represent a class of compounds that are inhibitors of HMG CoA reductase, a key enzyme in the cholesterol biosynthetic pathway (Endo, A., In: Cellular Metabolism of the Arterial Wall and Central Nervous System. Selected Aspects. Schettler G, Greten H, Habenicht A. J. R. (Eds.) Springer-Verlag, Heidelberg (1993)).

The statins decrease liver cholesterol biosynthesis, which increases the production of LDL receptors thereby decreasing total plasma and LDL cholesterol (Grundy, S. M. New Engl. J. Med. 319, 24 (1988); Endo, A., J. Lipid Res. 33, 1569 (1992)). Depending on the agent and the dose used, statins may decrease plasma triglyceride levels and some may increase HDLc. Currently the statins on the market are lovastatin (Merck), simvastatin (Merck), pravastatin (Sankyo and Squibb) and Fluvastatin (Sandoz). A fifth statin, atorvastatin (Parke-Davis/Pfizer), is the most recent entrant into the statin market. Statins have become the standard therapy for LDL cholesterol lowering. The statins are effective LDLc lowering agents but have some side effects, the most common being increases in serum enzymes (transaminases and creatinine kinase). In addition, these agents may also cause myopathy and rhabdomyolysis especially when combined with fibrates.

Another drug that in part may impact the liver is probucol (Zimetbaum, P., et al., Clin. Pharmacol. 30, 3 (1990)). Probucol is used primarily to lower serum cholesterol levels in hypercholesterolemic patients and is commonly administered in the form of tablets available under the trademark Lorelco™. Probucol is chemically related to the widely used food additives 2,[3]-tert-butyl4-hydroxyanisole (BHA) and 2,6-di-tert-butyl-4-methyl phenol (BHT). Its full chemical name is 4,4′-(isopropylidenedithio) bis(2,6-di-tert-butylphenol). Probucol is a lipid soluble agent used in the treatment of hypercholesterolemia including familial hypercholesterolemia (FH). Probucol reduces LDL cholesterol typically by 10% to 20%, and also reduces HDL by 20% to 30%. The drug has no effect on plasma triglycerides. The mechanism of action of probucol in lipid lowering is not completely understood. The LDLc lowering effect of probucol may be due to decreased production of apoB containing lipoproteins and increased clearance of LDL. Probucol lowers LDL in the LDL-receptor deficient animal model (WHHL rabbits) as well as in FH populations. Probucol has been shown to actually slow the progression of atherosclerosis in LDL receptor-deficient rabbits as discussed in Carew et al. (1987) Proc. Natl. Acad. Sci. U.S.A. 84:7725-7729. The HDL lowering effect of probucol may be due to decreased synthesis of HDL apolipoproteins and increased clearance of this lipoprotein. High doses of probucol are required in clinical use.

U.S. Pat. No. 6,004,936 to Robert Kisilevsky describes a method for potentiating the release and collection of cholesterol from inflammatory or atherosclerotic sites in vivo, the method including the steps of increasing the affinity of high-density lipoprotein for macrophages by administering to a patient an effective amount of a composition comprising a compound selected from the group consisting of native serum amyloid A (SAA) and a ligand having SAA properties thereby increasing the affinity of high density lipoprotein (HDL) for macrophages and potentiating release and collection of cholesterol.

U.S. Pat. No. 5,705,515 to Fisher; Michael H. et al.; U.S. Pat. No. 6,043,253 to Brockunier; Linda et al.; U.S. Pat. No. 6,034,106 to Biftu; Tesfaye et al.; and U.S. Pat. No. 6,011,048 to Mathvink; Robert J. et al. (Merck) describes substituted sulfonamides, fused piperidine substituted arylsulfonamides; oxadiazole substituted benzenesulfonamides and thiazole substituted benzenesulfonamides, respectively, as β₃ adrenergic receptor agonists with very little β₁ and β₂ adrenergic receptor activity as such the compounds are capable of increasing lipolysis and energy expenditure in cells. The compounds thus have potent activity in the treatment of Type II diabetes and obesity. The compounds can also be used to lower triglyceride levels and cholesterol levels or raise high density lipoprotein levels or to decrease gut motility. In addition, the compounds can be used to reduced neurogenic inflammation or as antidepressant agents. Compositions and methods for the use of the compounds in the treatment of diabetes and obesity and for lowering triglyceride levels and cholesterol levels or raising high density lipoprotein levels or for decreasing gut motility are also disclosed.

U.S. Pat. No. 5,120,766 to Holloway et al. describes the use of 2-(phenoxypropanolamino)ethoxyphenoxyacetic acid derivatives or a pharmaceutically acceptable salt thereof, in lowering triglyceride and/or cholesterol levels and/or increasing high density lipoprotein levels. These compounds are used in treating hypertriglycerdaemia, hyper-cholesterolaemia, conditions of low HDL (high density lipoprotein) levels and atherosclerotic disease.

U.S. Pat. No. 6,193,967 to Morganelli discloses bispecific molecules which react both with an Fcγ receptor for immunoglobulin G (IgG) of human effector cells and with either human low density lipoprotein (LDL), or fragment thereof, or human high density lipoprotein (HDL), or a fragment thereof. The bispecific molecules bind to a Fcγ receptor without being blocked by the binding of IgG to the same receptor. The bispecific molecules having a binding specificity for human LDL are useful for targeting human effector cells for degradation of LDL in vivo. The bispecific molecules of the '967 invention which have a binding specificity for human HDL are useful for targeting human HDL to human effector cells such that the HDL takes up cholesterol from the effector cells. Also disclosed are methods of treating atherosclerosis using these bispecific molecules.

U.S. Pat. No. 6,090,836 to Adams et al. discloses acetylphenols which are useful as antiobesity and antidiabetic compounds. Compositions and methods for the use of the compounds in the treatment of diabetes and obesity and for lowering or modulating triglyceride levels and cholesterol levels or raising high density lipoprotein levels or for increasing gut motility or for treating atherosclerosis.

U.S. Pat. No. 5,262,439 to Parthasarathy and assigned to AtheroGenics, Inc., discloses analogs of probucol with increased water solubility in which one or both of the hydroxyl groups are replaced with ester groups that increase the water solubility of the compound. In one embodiment, the derivative is selected from the group consisting of a mono- or di-probucol ester of succinic acid, glutaric acid, adipic acid, seberic acid, sebacic acid, azelaic acid or maleic acid. In another embodiment, the probucol derivative is a mono- or di-ester in which the ester contains an alkyl or alkenyl group that contains a functionality selected from the group consisting of a carboxylic acid group, amine group, salt of an amine group, amide groups, amide groups and aldehyde groups.

WO 98/09773 filed by AtheroGenics, Inc. discloses that monoesters of probucol, and in particular, the monosuccinic acid ester of probucol, are effective in simultaneously reducing LDLc, and inhibiting the expression of VCAM-1. These compounds are useful as composite cardiovascular agents. Since the compounds exhibits three important vascular protecting activities simultaneously, the patient can take one drug instead of multiple drugs to achieve the desired therapeutic effect.

De Meglio et al., have described several ethers of symmetrical molecules for the treatment of hyperlipidemia. These molecules contain two phenyl rings attached to each other through a —S—C(CH₃)₂—S— bridge. In contrast to probucol, the phenyl groups do not have t-butyl as substituents. (De Meglio et al., New Derivatives of Clofibrate and probucol: Preliminary Studies of Hypolipemic Activity; Farmaco, Ed. Sci (1985), 40 (11), 833-44).

WO 00/26184 discloses a large genus of compounds with a general formula of phenyl-S-alkylene-S-phenyl, in which one or both phenyl rings can be substituted at any position. These compounds were disclosed as lubricants.

A series of French patents disclose that certain probucol ester derivatives are hypocholesterolemic and hypolipemic agents: FR 2168137 (bis 4-hydroxyphenylthioalkane esters); FR 2140771 (tetralinyl phenoxy alkanoic esters of probucol); Fr 2140769 (benzofuryloxyalkanoic acid derivatives of probucol); FR 2134810 (bis-(3-alkyl-5-t-alkyl-4-thiazole-5-carboxy)phenylthio)alkanes; FR 2133024 (bis-(4-nicoinoyloxyphenythio)-propanes; and FR 2130975 (bis(4-(phenoxyalkanoyloxy)-phenylthio)alkanes).

U.S. Pat. No. 5,155,250 discloses that 2,6-dialkyl-4-silylphenols are anti-atherosclerotic agents. The same compounds are disclosed as serum cholesterol lowering agents in PCT Publication No. WO 95/15760, published on Jun. 15, 1995. U.S. Pat. No. 5,608,095 discloses that alkylated-4-silyl-phenols inhibit the peroxidation of LDL, lower plasma cholesterol, and inhibit the expression of VCAM-1, and thus are useful in the treatment of atherosclerosis.

U.S. Pat. No. 5,783,600 discloses that dialkyl ethers lower Lp(a) and triglycerides and elevate HDL-cholesterol and are useful in the treatment of vascular diseases.

A series of European patent applications of Shionogi Seiyaku Kabushiki Kaisha disclose phenolic thioethers for use in treating arteriosclerosis. European Patent Application No. 348 203 discloses phenolic thioethers that inhibit the denaturation of LDL and the incorporation of LDL by macrophages. The compounds are useful as anti-arteriosclerosis agents. Hydroxamic acid derivatives of these compounds are disclosed in European Patent Application No. 405 788 and are useful for the treatment of arteriosclerosis, ulcer, inflammation and allergy. Carbamoyl and cyano derivatives of the phenolic thioethers are disclosed in U.S. Pat. No. 4,954,514 to Kita, et al.

U.S. Pat. No. 4,752,616 to Hall, et al., discloses arylthioalkylphenylcarboxylic acids for the treatment of thrombotic disease. The compounds disclosed are useful as platelet aggregation inhibitors for the treatment of coronary or cerebral thromboses and the inhibition of bronchoconstriction, among others.

A series of patents to Adir et Compagnie disclose substituted phenoxyisobutyric acids and esters useful as antioxidants and hypolipemic agents. This series includes U.S. Pat. Nos. 5,206,247 and 5,627,205 to Regnier, et al. (which corresponds to European Patent Application No. 621 255) and European Patent Application No. 763 527.

WO 97/15546 to Nippon Shinyaku Co. Ltd. discloses carboxylic acid derivatives for the treatment of arterial sclerosis, ischemic heart diseases, cerebral infarction and post-PTCA restenosis.

The Dow Chemical Company is the assignee of patents to hypolipidemic 2-(3,5-di-tert-butyl-4-hydroxyphenyl)thio carboxamides. For example, U.S. Pat. Nos. 4,029,812, 4,076,841 and 4,078,084 to Wagner, et al., disclose these compounds for reducing blood serum lipids, especially cholesterol and triglyceride levels.

WO 98/51662 and WO 01/70757 filed by AtheroGenics, Inc., and U.S. Pat. No. 6,147,250 to AtheroGenics, Inc. disclose therapeutic agents for the treatment of diseases, including cardiovascular diseases, which are mediated by VCAM-1. One of these agents, designated as AGI 1067, a compound in development by AtheroGenics, Inc, is orally dosed once per day and has shown initial success in post-angioplasty restenosis. AGI 1067 may treat all areas of the coronary artery susceptible to atherosclerosis in a way that cannot be achieved with any existing therapy. Pre-clinical and early clincal testing of AGI 1067 has demonstrated that it blocks VCAM-1 expression, prevents atherosclerosis and shows potent anti-oxidant activity. Another agent, designated as AC 3056, a compound in development by Amlylin Pharmaceuticals, has been shown to reduce serum LDL, but not serum HDL, to inhibit lipoprotein oxidation, and to inhibit cell adhesion molecules in vascular cells. The data indicate that AC 3056 is an antioxidant that inhibits vascular cell adhesion molecule expression in human vascular cells. In animal models of atherosclerosis, AC 3056 is orally active, lowered serum cholesterol concentrations, inhibited the formation of atherosclerotic plaques in the arterial wall and prevented cholesterol-induced damage to vascular function.

Fatty Acid Synthesis

Plasma lipid levels may also be affected by cellular fatty acid synthesis which produces triacylglycerol and leads to the formation of VLDL. Fatty acid synthesis occurs in a relatively simple pathway in the cytoplasm of the cell and is dependent upon several crucial intermediates. The more important intermediates are citrate, a citric acid cycle component, and NADPH, a coenzyme generated from the action of malic enzyme and the pentose phosphate shunt. A key reaction involved in these pathways is the oxidative decarboxylation of L-malic acid to pyruvate by malic enzyme.

Malic acid is a naturally occurring compound, extracted in high yields from fruits, such as apples and pineapples (McKenzie et al, J. Chem. Soc. 123, 2875 (1923). Both the D- and L-isomers are found in these extracts. Although both isomers are found naturally, mammalian cells can only recognize the L-isomer of malic acid. The D-isomer is not utilized in triglyceride biosynthesis.

U.S. Pat. No. 2,972,566 to Kitahara discloses a process for the synthetic production of L-malic acid from fumerate using the enzyme fumerase. Fumerase can be obtained from various plants, animals and microorganisms including Lactobacillus or Escherichia coli. U.S. Pat. No. 3,063,910 to Abe et al discloses a method for the production of L-malic acid by fermentation using various species of Aspergillus.

U.S. Pat. No. 4,912,042 to Eastman Kodak Company and U.S. Pat. No. 5,824,449 to Ajinomoto Co., Inc. disclose methods for the production of D-malic acid from microorganisms. JP 2001/197897 to Mitshubishi Chemicals Corp. and JP 5271147 to Mitsubishi Petrochem Co. Ltd. disclose processes for the purification of D-malic acid. Both the D- and L-isomers and the D,L racemate can be obtained commercially (e.g. Sigma/Aldrich Chemicals).

Sicart and Samble-Amplis (Ann. Nutr. Metab. 31, 1 (1987) examined the influence of an apple-supplemented diet on the distribution of cholesterol among the lipoproteins in plasma in spontaneously hypercholesterolemic hamsters. They found that this diet decreased the cholesterol content in VLDL and LDL in plasma. However, the malic acid content of apples was not implicated in this effect.

Since cardiovascular disease is the leading cause of death in North America and in other industrialized nations, there is a need to provide new therapies for its treatment, especially treatments that work through a mechanism different from the current drugs and can be used in conjunction with them.

It is an object of the present invention to provide compounds, compositions, methods and uses that are useful to lower serum triglyceride, total cholesterol, LDL, and VLDL cholesterol levels.

It is another object of the present invention to provide a new method to improve the HDL/LDL ratio by lowering LDL levels to a greater extent than HDL levels.

### SUMMARY OF THE INVENTION

It has been discovered that administration of D-malic acid or its pharmaceutically acceptable salt, prodrug or pharmaceutically acceptable derivative can be used in the treatment or prevention of cardiovascular disease. In particular, it has been discovered that D-malic acid decreases serum triglyceride, total cholesterol, LDL, HDL and/or VLDL cholesterol levels.

In one aspect of the invention, a method for decreasing serum triglycerides, total cholesterol, LDL, and/or VLDL cholesterol levels in a host in need thereof, including a human, is provided that includes administering an effective amount of D-malic acid or its pharmaceutically acceptable salt, prodrug, or pharmaceutically acceptable derivative, optionally in a pharmaceutically acceptable carrier. In one embodiment, D-malic acid is in substantially pure form, essentially free of L-malic acid. In yet another embodiment, the D-malic acid can be administered as any D/L mixture including the racemate.

In one embodiment, the active compound agent decreases serum triglycerides, total cholesterol, LDL and VLDL cholesterol levels by at least 20 percent in a treated host, over the untreated serum level, and in a preferred embodiment, the compound decreases serum triglycerides, total cholesterol, LDL and VLDL cholesterol levels by at least 30, 40, 50, or 60 percent.

In yet another aspect, a method is provided for decreasing serum triglycerides, total cholesterol, LDL and/or VLDL cholesterol levels by administering a compound or a pharmaceutically acceptable prodrug of said compound, or a physiologically acceptable salt thereof, optionally in a pharmaceutically acceptable carrier, to a host in need thereof including a human, that includes administering an effective amount of a compound which interferes with fatty acid synthesis.

In still another aspect, assays are provided to identify compounds that decrease serum triglycerides, total cholesterol, LDL and VLDL cholesterol levels.

In an alternative aspect, a method is provided to decrease serum triglycerides, total cholesterol, LDL and VLDL cholesterol levels that includes administering D-malic acid or its pharmaceutically acceptable salt, prodrug or active derivative in combination or alternation with a lipid modulating compound, or, for example, with a compound selected from the group consisting of statins, IBAT inhibitors, MTP inhibitors, cholesterol absorption antagonists, phytosterols, CETP inhibitors, fibric acid derivatives and antihypertensive agents.

In an alternative aspect, a method is provided to decrease serum triglycerides, total cholesterol, LDL and VLDL cholesterol levels that includes administering D-malic acid or a pharmaceutically acceptable salt, prodrug or active derivative thereof, in combination or alternation with a lipid modulating compound that increases serum HDL levels.

In another aspect of the invention, D-malic acid or its pharmaceutically acceptable salt, prodrug or active derivative, optionally in a pharmaceutically acceptable carrier, is administered orally either alone, or in combination with another lipid lowering agent.

### BRIEF DESCRIPTION OF THE DRAWINGS

These figures form a part of the specification. It is to be noted, however, that the appended figures present illustrative embodiments of the invention and therefore are not to be considered limiting in their scope.

FIGS. 1A & 1B depict the amount of water consumption (ml/rat/day; A) and food consumption (g/day; B) over the course of the study described in Example 1; panel C depicts the ratio of Body weight (g)/age (weeks) of the rats during the course of the study. Treatment groups: controls—filled triangles; D-malic acid treated—x's; L-malic acid treated—filed squares; and D,L-malic acid treated—white triangles. Dosages are as described in Example 1.

FIGS. 2A & 2B depict the serum analysis from control, L, DL, and D-malic acid treated Zucker fa/fa rats from 6 to 24 weeks of age. Serum analysis included triglycerides, total cholesterol, HDL, aspartate amino transaminase (AST) and alanine amino transferaminase (ALT). Treatment groups: controls—filled triangles; D-malic acid treated—x's; L-malic acid treated—filed squares; and D,L-malic acid treated—white triangles. Dosages are as described in Example 1.

FIG. 3 depicts body weight plotted versus serum triglycerides (mg %) for control, L, DL, and D-malic acid treated Zucker fa/fa rats. The linear slope of control and L-malic acid treated rats differs significantly from the linear slope of D,L and D-malic acid treated rats (p≦0.01).

FIG. 4 shows the electrophroetic pattern of isoenzymes of cystolic malic enzyme, decarboxylating (1.1.1.40) illustrating the anodal Rf values in normal Sprague-Dawley (Normal), control Zucker rats (Control (Zucker)) and D-malic acid treated Zucker rats (D-malic acid treated (Zucker)).

FIG. 5 shows the percent oxygen consumption of mitochondria from a normal Sprague-Dawley rat in liver mitochondria following treatment with D-malic acid (D-malate, dashed line), varying L-malic acid with 20 μmoles D-malic acid (L-malate with 20 μmoles D-malate) and L-malic acid (L-malate).

FIG. 6 depicts the synthesis of short chain fatty acids occurs in the cytoplasm. Malic enzyme (Step 4) converts malic acid to pyruvate, which is shuttled back into the mitochondria. NADPH synthesized from malic enzyme is needed in the elongation of fatty acids during synthesis (from C. K. Mathews and K. E. Van Holde. Biochemistry. 2^{nd} ed. Benjamin Cummings Pub. Co.).

### DETAILED DESCRIPTION OF THE INVENTION

It has been discovered that D-malic acid or its pharmaceutically acceptable salt, prodrug or active derivative (“active compound”) is useful for decreasing lipoprotein cholesterol, triglycerides and total serum cholesterol by interfering with fatty acid synthesis.

It has been discovered that these compounds significantly decrease LDL, HDL, VLDL, total serum triglycerides and/or cholesterol levels.

In one embodiment of the invention, a method for decreasing serum triglycerides, total cholesterol, LDL and/or VLDL cholesterol levels in a host in need thereof, including a human, is provided that includes administering an effective amount of D-malic acid or a pharmaceutically acceptable salt, prodrug or active derivative thereof, optionally in a pharmaceutically acceptable carrier.

In another embodiment, the active agent decreases serum triglycerides, total cholesterol, LDL and VLDL cholesterol levels by at least 20 percent in a treated host (for example, an animal, including a human), over the untreated serum levels, and in a preferred embodiment, the compound decreases serum triglycerides, total cholesterol, LDL and VLDL cholesterol levels by at least 30, 40, 50, or 60 percent.

In still another embodiment, assays are provided to identify compounds that decrease circulating lipoprotein cholesterol levels or decrease total triglyceride levels.

In an alternative embodiment, a method is provided to decrease serum lipoproteins that includes administering D-malic acid, or a pharmaceutically acceptable salt or prodrug thereof, optionally in a pharmaceutically acceptable carrier, in combination or alternation with a lipid modulating compound, or, for example, with a compound selected from the group consisting of statins, IBAT inhibitors, MTP inhibitors, cholesterol absorption antagonists, phytosterols, CETP inhibitors, fibric acid derivatives and antihypertensive agents.

In another embodiment, a method is provided to decrease serum triglycerides, total cholesterol, LDL and VLDL cholesterol levels that includes administering D-malic acid or a pharmaceutically acceptable salt or prodrug thereof, in combination or alternation with a lipid modulating compound that increases serum HDL levels.

In another embodiment of the invention, a method for determining whether a compound will decrease serum triglycerides, total cholesterol, LDL and VLDL cholesterol levels is provided that includes assaying the ability of the compound to form a complex with a malic enzyme and then assessing whether the newly formed complex inhibits the oxidative decarboxylation of malic acid to pyruvate, thereby decreasing serum triglycerides, total cholesterol, LDL and VLDL cholesterol levels.

As one nonlimiting example of this embodiment, a method is provided comprising, a) contacting a test compound with malic enzyme; b) contacting an animal model, or alternatively a cell line, with the combination of test compound with malic enzyme; c) determining the level of pyruvate accumulation; d) comparing the levels of pyruvate accumulation in a treated animal or cell model with an animal or cell model not contacted with the test compound; e) selecting the compound wherein there is a substantial decrease in pyruvate formation.

As another nonlimiting example, a method is provided comprising, a) administering a test compound to an animal model over a period of time, preferably six weeks; b) monitoring the level of serum LDL; c) monitoring the level of HDL; d) comparing the levels of LDL and HDL in the animal model in which the compound was administered with the levels of LDL and HDL in an animal model in which the compound was not administered; f) selecting the compound wherein there is a substantial decrease in HDL and LDL levels; g) selecting compounds which improve lipoprotein levels by assessing the ratio of HDL/LDL present in the blood of an animal model.

As one nonlimiting example of this embodiment, the test compound can be fed to a host animal, for example a rabbit, together with a high-fat diet for six weeks at a suitable dosage orally. The animals are then bled, preferably at six weeks, and lipoproteins isolated using high speed ultra-centrifugation. The amount of test compound bound to malic enzyme is then estimated.

I. Active Compound

By the “active compound” or “agent” is meant a compound of the formula:

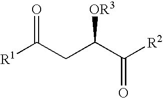

or a pharmaceutically acceptable salt or prodrug thereof, wherein:

R¹ and R² are independently any group that does not otherwise adversely affect the desired properties of the molecule, and for example includes but is not limited to OR⁴, alkyl, substituted alkyl, alkenyl, substituted alkenyl, alkynyl, substituted alkynyl, alkoxy, substituted alkyloxy, alkoxyalkyl, substituted alkoxyalkyl, NH₂, NHR⁵, NR⁷R⁶, mono- or polyhydroxy-substituted alkyl, aryl, substituted aryl, heteroaryl, substituted heteroaryl, acyloxy, substituted acyloxy, or haloalkyl, including CF₃;

R³ is any group that does not otherwise adversely affect the desired properties of the molecule, and for example includes but is not limited to hydrogen, alkyl, substituted alkyl, alkenyl, substituted alkenyl, alkynyl, substituted alkynyl, alkoxy, substituted alkyloxy, alkoxyalkyl, substituted alkoxyalkyl, mono- or polyhydroxy-substituted alkyl, aryl, substituted aryl, heteroaryl, substituted heteroaryl, acyloxy, substituted acyloxy, alkylsulfonyl, arylsulfonyl, aralkylsulfonyl, amino acid residue, haloalkyl, including CF3, or the carboxylic moiety of an ester, including CO-alkyl, CO-aryl, CO-alkoxyalkyl, CO-aryloxyalkyl, CO-substituted aryl.

R⁴ is any group that does not otherwise adversely affect the desired properties of the molecule, for example includes but is not limited to hydrogen, alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy.

R⁵, R⁶, and R⁷ are independently any group that does not otherwise adversely affect the desired properties of the molecule, and for example includes but is not limited to alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy.

II. Definitions

The term alkyl, as used herein, unless otherwise specified, refers to a saturated straight, branched, or cyclic, primary, secondary, or tertiary hydrocarbon, including but not limited to those of C₁ to C₁₀, and preferably C₁-C₆, including methyl, (cyclopropyl)methyl, (cyclobutyl)methyl, (cyclopentyl)methyl, ethyl, 1-cyclopropylethyl, 2-cyclopropylethyl, 1-cyclobutylethyl, 2-cyclobutylethyl, propyl, isopropyl, 1-(cyclopropyl)propyl, 2-(cyclopropyl)propyl, 3-(cyclopropyl)propyl, cyclopropyl, methylcyclopropyl, 2,2-dimethylcyclopropyl, 1,2-dimethylcyclopropyl, ethylcyclopropyl, propylcyclopropyl, 1-ethyl-1-methylcyclopropyl, 1-ethyl-2-methylcyclopropyl, 1,1,2-trimethylcyclopropyl, 1,2,3-trimethylcyclopropyl, butyl, isobutyl, t-butyl, sec-butyl, 2,2-dimethylbutyl, 2,3-dimethylbutyl, cyclobutyl, methylcyclobutyl, 1,1-dimethylcyclobutyl, 1,2-dimethylcyclobutyl, 1,3-dimethylcyclobutyl, ethylcyclobutyl, pentyl, isopentyl, neopentyl, 2-methylpentyl, 3-methylpentyl, cyclopentyl, methylcyclopentyl, spiropentyl, methylspiropentyl, hexyl, isohexyl and cyclohexyl. The alkyl group can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, thiol, imine, sulfonic acid, sulfate, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphate, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991.

The term aryl, as used herein, and unless otherwise specified, refers to phenyl, biphenyl, or naphthyl, and preferably phenyl. The aryl group can be optionally substituted with one or more of the moieties selected from the group consisting of alkyl, heteroaryl, heterocyclic, carbocycle, alkoxy, aryloxy, aryloxy; arylalkoxy; heteroaryloxy; heteroarylalkoxy, carbohydrate, amino acid, amino acid esters, amino acid amides, alditol, halo, haloalkyl, hydroxyl, carboxyl, acyl, acyloxy, amino, amido, alkylamino, dialkylamino, arylamino, nitro, cyano, thiol, imide, sulfonic acid, sulfate, sulfonyl, sulfanyl, sulfinyl, sulfamoyl, carboxylic ester, carboxylic acid, amide, phosphonyl, phosphinyl, phosphoryl, thioester, thioether, oxime, hydrazine, carbamate, phosphonic acid, phosphate, phosphonate, phosphinate, sulfonamido, carboxamido, hydroxamic acid, sulfonylimide or any other desired functional group that does not inhibit the pharmacological activity of this compound, either unprotected, or protected as necessary, as known to those skilled in the art, for example, as taught in Greene, et al., “Protective Groups in Organic Synthesis,” John Wiley and Sons, Second Edition, 1991. Alternatively, adjacent groups on the aryl ring may combine to form a 5 to 7 membered carbocyclic, aryl, heteroaryl or heterocyclic ring. In another embodiment, the aryl ring is substituted with an optionally substituted cycloalkyl (such as cyclopentyl or cyclohexyl), or an alkylene dioxy moiety (for example methylenedioxy).

The term “heteroaryl or heteroaromatic,” as used herein, refers to an aromatic that includes at least one sulfur, oxygen, nitrogen or phosphorus in the aromatic ring. The term “heterocyclic” refers to a nonaromatic cyclic group wherein there is at least one heteroatom, such as oxygen, sulfur, nitrogen or phosphorus in the ring. Nonlimiting examples of heteroaryl and heterocyclic groups include pyrimidines, such as thymine, cytosine and uracil, substituted pyrimidines such as N5-halopyrimidines, N5-alkylpyrimidines, N5-benzylpyrimidines, N5-vinylpyrimidine, N5-acetylenic pyrimidine, N5-acyl pyrimidine, 6-azapyrimidine, 2-mercaptopyrmidine, and in particular, 5-fluorocytidinyl, 5-azacytidinyl, 5-azauracilyl, purines such as adenine, guanine, inosine and pteridine, substituted purines such as N6-alkylpurines, N6-benzylpurine, N6-halopurine, N6-vinypurine, N6-acetylenic purine, N6-acyl purine, N6-thioalkyl purine, N6-hydroxyalkyl purine, N6-thioalkyl purine and N5-hydroxyalkyl purine and in particular, 6-chloroadenine and 6-azoadenine, triazolopyridinyl, imidazolopyridinyl, pyrrolopyrimidinyl, pyrazolopyrimidinyl, pyridine, pyrrole, indole, imidazole, pyrazole, quinazoline, pyridazine, pyrazine, cinnoline, phthalazine, quinoxaline, xanthine, hypoxanthine, triazolopyridine, imidazolepyridine, imidazolotriazine, pyrrolopyrimidine, pyrazolopyrimidine, 1-triphenyl-methyltetrazolyl, 2-triphenylmethyl-tetrazolyl group, fliryl, furanyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, oxazole, thiazole, isothiazole, pyridazine, and pteridinyl, aziridines, thiazole, 1,2,3-oxadiazole, thiazine, pyridine, pyrazine, piperazine, pyrrolidine, oxaziranes, phenazine, phenothiazine, morpholinyl, pyrazolyl, pyridazinyl, pyrazinyl, quinoxalinyl, xanthinyl, hypoxanthinyl, pteridinyl, isoxazolyl, pyrrolidin-2-yl, piperidin-2-yl, quinolin-2-yl, isoquinolin-1-yl, pyridin-2-yl, 4-methylimidazol-2-yl, 1-methylimidazol-4-yl, 1-n-hexylimidazol-4-yl, 1-benzylimidazol-4-yl, 1,2-dimethylimidazol-4-yl, 1-n-pentyl-2-methyl-imidazol-4-yl, 1-benzyl-2-methyl-imidazol-5-yl, benzimidazol-2-yl, 1-methylbenzimidazol-2-yl, 1-methyl-5-methoxy-benzimidazol-2-yl, imidazo[1,2-a]pyridin-2-yl, 6-chloro-imidazo [1,2-a]-pyridin-2-yl, imidazo[1,2-a]pyrimidin-2-yl, 2-phenyl-imidazo[2,1-b]-thiazol-6-yl, purin-8-yl, imidazo[4,5-b]pyrazin-2-yl, 5-methyl-imidazolidin-2,4-dion-3-yl, 2-n-propyl-pyridazin-3-on-6-yl, oxazol-4-yl, 2-isopropyl-thiazol-4-yl, 1-ethyl-imidazol-4-yl, 1-(4-fluorobenzyl)-2-methyl-imidazol-4-yl, 1-minocarbonylmethyl-imidazol-4-yl, 1-morpholino-carbonylmethyl-imidazol-4-yl, 2-isopropyl-pyridazin-3-on-6-yl, 2-benzyl-pyridazin-3-on-6-yl, 2-(2-phenylethyl)-pyridazin-3-on-6-yl, 2˜(3˜phenylpropyl)-pyridazin-3-on-6-yl, 4-methyl-pyridazin-3-on-6-yl, 5-methyl-pyridazin-3-on-6-yl, 4,5-dimethyl-pyridazin-3-on-6-yl, 2,4-dimethyl-pyridazin-3-on-6-yl, 2,5-dimethyl-pyridazin-3-on-6-yl, 2,4,5-trimethyl-pyridazin-3-on-6-yl. The heteroaromatic group can be optionally substituted as described above for aryl. The heterocyclic group can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991. The heteroaromatic can be partially or totally hydrogenated as desired. As a nonlimiting example, dihydropyridine can be used in place of pyridine. Functional oxygen and nitrogen groups on the heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenesulfonyl.

The term aralkyl, as used herein, and unless otherwise specified, refers to an aryl group as defined above linked to the molecule through an alkyl group as defined above. The term alkaryl, as used herein, and unless otherwise specified, refers to an alkyl group as defined above linked to the molecule through an aryl group as defined above. The aralkyl or alkaryl group can be optionally substituted with one or more moieties selected from the group consisting of hydroxyl, acyl, amino, alkylamino, arylamino, alkoxy, aryloxy, nitro, cyano, sulfonic acid, sulfate, phophonic acid, phosphate, or phosphonate, either unprotected, or protected as necessary, as known to those skilled in the art, for example, as taught in Greene, et al., 1991.

The term halo, as used herein, includes chloro, bromo, iodo, and fluoro.

The term alkoxy, as used herein, and unless otherwise specified, refers to a moiety of the structure —O-alkyl, wherein alkyl is as defined above.

The term acyl, as used herein, refers to a group of the formula C(O)R′, wherein R′ is an alkyl, aryl, alkaryl or aralkyl group, or substituted alkyl, aryl, aralkyl or alkaryl, wherein these groups are as defined above.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including, sulfate, nitrate, bicarbonate, and carbonate salts.

“Pharmaceutically acceptable salts or complexes” refers to salts or complexes that retain the desired biological activity of the compounds of the present invention and exhibit minimal undesired toxicological effects. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, ascorbic acid, benzoic acid, tannic acid, pamoic acid, alginic acid, polyglutamic acid, naphthalenesulfonic acid, naphthalenedisulfonic acid, and polygalcturonic acid; (b) base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylenediamine, D-glucosamine, tetraethylammonium, or ethylenediamine; or (c) combinations of (a) and (b); e.g., a zinc tannate salt or the like. Also included in this definition are pharmaceutically acceptable quaternary salts known by those skilled in the art, which specifically include the quaternary ammonium salt of the formula —NR⁺A⁻, wherein R is as defined above and A is a counterion, including chloride, bromide, iodide, —O-alkyl, toluenesulfonate, methylsulfonate, sulfonate, phosphate, or carboxylate (such as benzoate, succinate, acetate, glycolate, maleate, malic acid, citrate, tartrate, ascorbate, benzoate, cinnamoate, mandeloate, benzyloate, and diphenylacetate).

The term “lipoprotein” refers to proteins that transport lipids including chylomicrons, very low density lipoproteins (VLDL), low density lipoproteins (LDL), high density lipoproteins (HDL), apolipoproteins (such as apoAI), or other proteins which complex with lipids.

The term “host,” as used herein, refers to any bone-containing animal, including, but not limited to humans, other mammals, mice, rats, rabbits, ferrets, pigs, canines, equines, felines, bovines, birds (such as chickens, turkeys, and other meat producing birds), cows, and bulls.

The term “lipid modulating agent” or “lipoprotein lowering agent” refers to an agent that lowers serum trigylcerides, total cholesterol, LDL, VLDL or HDL.

The term “prodrug,” as used herein, refers to any compound which, upon administration to a host, is converted or metabolized to an active compound described herein.

III. Stereochemistry

The present invention is based on the discovery that D-malic acid has useful properties in the treatment of cardiovascular disorders or hyperlipidemia, while L-malic acid is a natural component of fatty acid synthesis. Therefore, it is important according to the invention to provide the active compound in the form of the D-stereoisomer of malic acid. If substituent groups other than hydrogen are in the R¹, R², or R³ positions, and the substituent is chiral, it can be used in any desired stereochemical form that achieves the desired results. It is thus to be understood that the present invention encompasses any racemic, optically-active, polymorphic, or stereoisomeric form, or mixtures thereof, of a compound of the invention, which possess the useful properties described herein. It is known in the art how to prepare optically active forms and how to determine activity using the standard tests described herein, or using other similar tests which are well known in the art. Examples of methods that can be used to obtain optical isomers of the compounds of the present invention include the following.

i) physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization—a technique whereby the individual enantiomers are separately crystallized from a solution of the racemate, possible only if the latter is a conglomerate in the solid state;

iii) enzymatic resolutions—a technique whereby partial or complete separation of a racemate by virtue of differing rates of reaction for the enantiomers with an enzyme

iv) enzymatic asymmetric synthesis—a synthetic technique whereby at least one step of the synthesis uses an enzymatic reaction to obtain an enatiomerically pure or enriched synthetic precursor of the desired enantiomer;

v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;

vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors—a synthetic technique whereby the desired enantiomer is obtained from non-chiral starting materials and where the stereochemical integrity is not or is only minimally compromised over the course of the synthesis;

x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;

xi) chiral gas chromatography—a technique whereby the racemate is volatilized and enantiomers are separated by virtue of their differing interactions in the gaseous mobile phase with a column containing a fixed non-racemic chiral adsorbent phase;

xii) extraction with chiral solvents—a technique whereby the enantiomers are separated by virtue of preferential dissolution of one enantiomer into a particular chiral solvent;

xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

IV. Pharmaceutical Compositions

Animals, particularly mammal, and more particularly, humans, equine, canine or bovine can be treated for any of the conditions described herein by administering to the subject an effective amount of one or more of the above-identified compounds or a pharmaceutically acceptable prodrug or salt thereof in a pharmaceutically acceptable carrier or dilutant. Any appropriate route can be used to administer the active materials, for example, orally, parenterally, intravenously, intradermally, subcutaneously or topically.

The active compound is included in the pharmaceutically acceptable carrier or diluent in an amount sufficient to deliver to a patient a therapeutically effective amount without causing serious toxic effects in the patient treated. A preferred dose of the active compound for all of the above-mentioned conditions is in the range from about 0.1 to 500 mg/kg, preferably 1 to 100 mg/kg per day. The effective dosage range of the pharmaceutically acceptable prodrugs can be calculated based on the weight of the parent compound to be delivered. If the derivative exhibits activity in itself, the effective dosage can be estimated as above using the weight of the derivative, or by other means known to those skilled in the art.

For systemic administration, the compound is conveniently administered in any suitable unit dosage form, including but not limited to one containing 1 to 5000 mg, preferably 5 to 1000 mg of active ingredient per unit dosage form. An oral dosage of 25-3500 mg is usually convenient. The active ingredient should be administered to achieve peak plasma concentrations of the active compound of about 0.1 to 100 mM, preferably about 1-10 mM. This may be achieved, for example, by the intravenous injection of a solution or formulation of the active ingredient, optionally in saline, or an aqueous medium or administered as a bolus of the active ingredient.

The concentration of active compound in the drug composition will depend on absorption, distribution, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The active compound or pharmaceutically acceptable salt or derivative thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable prodrugs or salts thereof can also be administered with other active materials that do not impair the desired action, or with materials that supplement the desired action, such as antibiotics, antifungals, antiinflammatories, or antiviral compounds. The active compounds can be administered with lipid lowering agents such as probucol and nicotinic acid; platelet aggregation inhibitors such as aspirin; antithrombotic agents such as coumadin; calcium channel blockers such as varapamil, diltiazem, and nifedipine; angiotensin converting enzyme (ACE) inhibitors such as captopril and enalopril, and β-blockers such as propanalol, terbutalol, and labetalol. The compounds can also be administered in combination with nonsteroidal antiinflammatories such as ibuprofen, indomethacin, aspirin, fenoprofen, mefenamic acid, flufenamic acid, sulindac. The compound can also be administered with corticosteriods.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

Suitable vehicles or carriers for topical application are known, and include lotions, suspensions, ointments, creams, gels, tinctures, sprays, powders, pastes, slow-release transdermal patches, aerosols for asthma, and suppositories for application to rectal, vaginal, nasal or oral mucosa.

Thickening agents, emollients and stabilizers can be used to prepare topical compositions. Examples of thickening agents include petrolatum, beeswax, xanthan gum or polyethylene glycol, humectants such as sorbitol, emollients such as mineral oil, lanolin and its derivatives, or squalene. A number of solutions and ointments are commercially available.

Natural or artificial flavorings or sweeteners can be added to enhance the taste of topical preparations applied for local effect to mucosal surfaces. Inert dyes or colors can be added, particularly in the case of preparations designed for application to oral mucosal surfaces.

The active compounds can be prepared with carriers that protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylacetic acid. Many methods for the preparation of such formulations are patented or generally known to those skilled in the art.

If administered intravenously, preferred carriers are physiological saline or phosphate buffered saline (PBS).

The active compound can also be administered through a transdermal patch. Methods for preparing transdermal patches are known to those skilled in the art. For example, see Brown, L., and Langer, R., Transdermal Delivery of Drugs, Annual Review of Medicine, 39:221-229 (1988).

In another embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylacetic acid. Methods for preparation of such formulations will be apparent to those skilled in the art. The materials can also be obtained commercially from Alza Corporation and Nova Pharmaceuticals, Inc. Liposomal suspensions may also be pharmaceutically acceptable carriers. These may be prepared according to methods known to those skilled in the art, for example, as described in U.S. Pat. No. 4,522,811. For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives are then introduced into the container. The container is then swirled by hand to free lipid material from the sides of the container and to disperse lipid aggregates, thereby forming the liposomal suspension.

V. Combination and Alternation Therapy

The active compound of the present invention can be combined or alternated with other biologically active compounds to achieve a number of potential objectives. For example, through dosage adjustment and medical monitoring, the individual dosages of the therapeutic compounds used in the combinations of the present invention will be lower than are typical for dosages of the therapeutic compounds when used in monotherapy. The dosage lowering will provide advantages including reduction of side effects of the individual therapeutic compounds when compared to the monotherapy. In addition, fewer side effects of the combination therapy compared with the monotherapies will lead to greater patient compliance with therapy regimens.

Another use of the present invention will be in combinations having complementary effects or complementary modes of action. Compounds of the present invention can be administered in combination with a drug that lowers cholesterol via a different biological pathway, to provide augmented results.

The compounds of the present invention have been found to decrease serum concentrations of HDL. Since increased HDL levels have been shown to be an indicator in the beneficial effects of lipid lowering agents, still another use of the present invention is in combinations with drugs which increase levels of HDL.

Compounds useful for combining with the compounds of the present invention encompass a wide range of therapeutic compounds. IBAT inhibitors, for example, are useful in the present invention, and are disclosed in patent application no. PCT/US95/10863. More IBAT inhibitors are described in PCT/US97/04076. Still further IBAT inhibitors useful in the present invention are described in U.S. application Ser. No. 08/816,065. More IBAT inhibitor compounds useful in the present invention are described in WO 98/40375, and WO 00/38725. Additional IBAT inhibitor compounds useful in the present invention are described in U.S. application Ser. No. 08/816,065.

In another aspect, the second cholesterol lowering agent is a statin. The combination of the a fatty acid synthesis inhibiting drug with a statin creates a synergistic or augmented lowering of serum cholesterol, because statins lower cholesterol by a different mechanism, i.e., by inhibiting of 3-hydroxy-3-methylglutaryl coenzyme A (HMG CoA) reductase, a key enzyme in the cholesterol biosynthetic pathway. The statins decrease liver cholesterol biosynthesis, which increases the production of LDL receptors thereby decreasing plasma total and LDL cholesterol (Grundy, S. M. New Engl. J. Med. 319, 24 (1988); Endo, A. J. Lipid Res. 33, 1569 (1992)). Depending on the agent and the dose used, statins may decrease plasma triglyceride levels and may increase HDL. Currently the statins on the market are lovastatin (Merck), simvastatin (Merck), pravastatin (Sankyo and Squibb) and fluvastatin (Sandoz). A fifth statin, atorvastatin (Parke-Davis/Pfizer), is the most recent entrant into the statin market.

The following list discloses these preferred statins and their preferred dosage ranges.

TABLE 1

| | | | Normal | |
|---|---|---|---|---|
| | Trade | Dosage range | dose | Patent |
| | name | (mg/d) | (mg/d) | Reference |
| Fungal derivatives | | | | |
| lovastatin | Mevacor | 10-80 | 20-40 | 4,231,938 |
| pravastatin | Pravachol | 10-40 | 20-40 | 4,346,227 |
| simvastatin | Zocor | 5-40 | 5-10 | 4,739,073 |
| Synthetic compound | | | | |
| Fluvastatin | Lescol | 20-80 | 20-40 | 4,739,073 |

The following list describes the chemical formula of some preferred statins:

lovastatin: [1S[1a(R),3 alpha,7 beta,8 beta(2S,4S),8a beta]]-1,2,3,7,8,8a-hexahydro-3,7-dimethyl-8-[2-(tetrahydro-4-hydroxy-6-oxo-2H-pyran-2-yl)ethyl]-1-maphthalenyl-2-methylbutanoate

pravastatin sodium: 1-Naphthalene-heptanoic acid, 1,2,6,7,8a-hexahydro-beta,delta,6-trihydroxy-2-methyl-8-(2-ethyl-1-oxybutoxy)-1-, monosodium salt [1S-[1 alpha (beta s,delta S),2 alpha,6 alpha,8 beta (R),8a alpha

simvastatin: butanoic acid, 2,2-dimethyl-,1,2,3,7,8,8a-hexahydro-3,7-dimethyl-8-[2 tetrahydro-4-hydroxy-6-oxo-2H-pyran-2-yl)ethyl]-1-napthalenyl ester [1S-[1 alpha,3 alpha,7 beta,8 beta,(2S,4S),-8a beta

sodium fluvastatin: [R,S-(E)]-(+/−)-7-[3(4-fluorophenyl)-1-(1-methylethyl)-1H-indol-2-yl]-3,5-dihydroxy-6-heptenoic acid, monosodium salt

Other statins, and references from which their description can be derived, are listed below.

TABLE 2

| STATIN | REFERENCE |
|---|---|
| Atorvastatin | U.S. Pat. No. 5,273,995 |
| Cerivastatin (Baycol) | U.S. Pat. No. 5,177,080 |
| Mevastatin | U.S. Pat. No. 3,983,140 |
| Cerivastatin | U.S. Pat. No. 5,502,199 |
| Velostatin | U.S. Pat. No. 4,448,784 |
| Compactin | U.S. Pat. No. 4,804,770 |
| Dalvastatin | EP 738510 A2 |
| Fluindostatin | EP 363934 A1 |
| Dihydorcompactin | U.S. Pat. No. 4,450,171 |

Other statins include rivastatin, SDZ-63,370 (Sandoz), CI-981 (W-L). HR-780, L-645,164, CL-274,471, alpha-, beta-, and gamma-tocotrienol, (3R,5S,6E)-9,9-bis(4-fluoro-phenyl)-3,5-dihydroxy-8-(1-methyl-1H-tetrazol-5-yl)-6,8-nonadienoic acid, L-arginine salt, (S)-4-[[2-[4-(4-fluorophenyl)-5-methyl-2-(1-methylethyl)-6-phenyl-3-pyridinyl]ethenyl]-hydroxyphosphinyl]-3-hydroxybutanoic acid, disodium salt, BB-476, (British Biotechnology), dihydrocompactin, [4R-[4 alpha,6 beta(E)]]-6-[2-[5-(4-fluorophenyl)-3-(1-methylethyl)-1-(2-pyridinyl)-1H-pyrazol-4-yl]ethenyl]tetrahydro-4-hydroxy-2H-pyran-2-one, and 1H-pyrrole-1-heptanoic acid, 2-(4-fluorophenyl)-beta,delta-dihydroxy-5-(1-methylethyl)-3-phenyl-4-[(phenylamino)carbonyl]calcium salt[R-(R*,R*)].

However, the invention should not be considered to be limited to the foregoing statins. Naturally occurring statins are derivatives of fungi metabolites (ML-236B/compactin/monocalin K) isolated from Pythium ultimum, Monacus ruber, Penicillium citrinum, Penicillium brevicompactum and Aspergillus terreus, though as shown above they can be prepared synthetically as well. Statin derivatives are well known in the literature and can be prepared by methods disclosed in U.S. Pat. No. 4,397,786. Other methods are cited in The Peptides: Vol. 5, Analysis, Synthesis, Biology; Academic Press NY (1983); and by Bringmann et al. in Synlett (5), pp. 253-255 (1990).

Thus, the term statin as used herein includes any naturally occurring or synthetic peptide that inhibits 3-hydroxy-3-methylglutaryl coenzyme A (HMG CoA) reductase by competing with 3-hydroxy-3-methylglutaric acid (HMG) CoA for the substrate binding site on HMG-CoA reductase. Assays for determining whether a statin acts through this biological pathway are disclosed in U.S. Pat. No. 4,231,938, column 6, and WO 84/02131 on pages 30-33.

MTP inhibitor compounds useful in the combinations and methods of the present invention comprise a wide variety of structures and functionalities. Some of the MTP inhibitor compounds of particular interest for use in the present invention are disclosed in WO 00/38725. Descriptions of these therapeutic compounds can be found in Science, 282, 23 Oct. 1998, pp. 751-754.

Cholesterol absorption antagonist compounds useful in the combinations and methods of the present invention comprise a wide variety of structures and functionalities. Some of the cholesterol absorption antagonist compounds of particular interest for use in the present invention are described in U.S. Pat. No. 5,767,115. Further cholesterol absorption antagonist compounds of particular interest for use in the present invention, and methods for making such cholesterol absorption antagonist compounds are described in U.S. Pat. No. 5,631,365.

A number of phytosterols suitable for the combination therapies of the present invention are described by Ling and Jones in “Dietary Phytosterols: A Review of Metabolism, Benefits and Side Effects,” Life Sciences, 57 (3), 195-206 (1995). Without limitation, some phytosterols of particular use in the combination of the present invention are Clofibrate, Fenofibrate, Ciprofibrate, Bezafibrate, Gemfibrozil. The structures of the foregoing compounds can be found in WO 00/38725.

Phytosterols are also referred to generally by Nes (Physiology and Biochemistry of Sterols, American Oil Chemists' Society, Champaign, Ill., 1991, Table 7-2). Especially preferred among the phytosterols for use in the combinations of the present invention are saturated phytosterols or stanols. Additional stanols are also described by Nes (Id.) and are useful in the combination of the present invention. In the combination of the present invention, the phytosterol preferably comprises a stanol. In one preferred embodiment the stanol is campestanol. In another preferred embodiment the stanol is cholestanol. In another preferred embodiment the stanol is clionastanol. In another preferred embodiment the stanol is coprostanol. In another preferred embodiment the stanol is 22,23-dihydrobrassicastanol. In another embodiment the stanol is epicholestanol. In another preferred embodiment the stanol is fucostanol. In another preferred embodiment the stanol is stigmastanol.

In another embodiment the present invention encompasses a therapeutic combination of a compound of the present invention and an HDL elevating agent. In one aspect, the HDL elevating agent can be a CETP inhibitor. Individual CETP inhibitor compounds useful in the present invention are separately described in WO 00/38725. Other individual CETP inhibitor compounds useful in the present invention are separately described in WO 99/14174, EP818448, WO 99/15504, WO 99/14215, WO 98/04528, and WO 00/17166. Other individual CETP inhibitor compounds useful in the present invention are separately described in WO 00/18724, WO 00/18723, and WO 00/18721. Other individual CETP inhibitor compounds useful in the present invention are separately described in WO 98/35937. Particular CETP inhibitors suitable for use in combination with the invention are described in The Discovery of New Cholesteryl Ester Transfer Protein Inhibitors (Sikorski et al., Curr. Opin. Drug Disc. & Dev., 4(5):602-613 (2001)).

Of particular interest as CETP inhibitors are the compounds disclosed in U.S. Pat. Nos. 6,197,786 and 6,313,142. Specifically, the compound (−)(2R,4S)-4-Amino-2-2-ethyl-6-trifluoromethyl-3,4-dihydro-2H-quinoline-1-carboxylicacid ethyl ester and its salts is disclosed. Said compound having the formula:

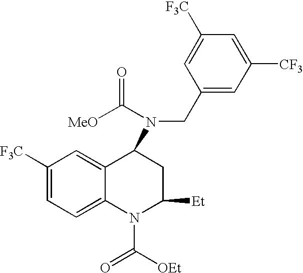

In another aspect, the HDL elevating agent can be a fibric acid derivative. Fibric acid derivatives useful in the combinations and methods of the present invention comprise a wide variety of structures and functionalities. Preferred fibric acid derivatives for the present invention are described in Table 3. The therapeutic compounds of Table 3 can be used in the present invention in a variety of forms, including acid form, salt form, racemates, enantiomers, zwitterions, and tautomers.

TABLE 3

| | | U.S. Pat. |
|---|---|---|
| | CAS Registry | Reference for |
| Common Name | Number | Compound Per Se |
| Clofibrate | 637-07-0 | 3,262,850 |
| Fenofibrate | 49562-28-9 | 4,058,552 |
| Ciprofibrate | 52214-84-3 | 3,948,973 |
| Bezafibrate | 41859-67-0 | 3,781,328 |
| Gemfibrozil | 25182-30-1 | 3,674,836 |

In another embodiment the present invention encompasses a therapeutic combination of a compound of the present invention and an antihypertensive agent. Hypertension is defined as persistently high blood pressure. Generally, adults are classified as being hypertensive when systolic blood pressure is persistently above 140 mmHg or when diastolic blood pressure is above 90 mmHg. Long-term risks for cardiovascular mortality increase in a direct relationship with persistent blood pressure (E. Braunwald, Heart Disease, 5^{th} ed., W. B. Saunders & Co., Philadelphia, 1997, pp. 807-823) Blood pressure is a function of cardiac output and peripheral resistance of the vascular system and can be represented by the following equation:

BP=CO×PR

wherein BP is blood pressure, CO is cardiac output, and PR is peripheral resistance (E. Braunwald, Heart Disease, 5^{th} ed., W. B. Saunders & Co., Philadelphia, 1997, pp. 807-823). Factors affecting peripheral resistance include obesity and/or functional constriction. Factors affecting cardiac output include venous constriction. Functional constriction of the blood vessels can be caused y a variety of factors including thickening of blood vessel walls resulting in diminishment of the inside diameter of the vessels. Another factor which affects systolic blood pressure is rigidity of the aorta (E. Braunwald, Heart Disease, 5^{th} ed., W. B. Saunders & Co., Philadelphia, 1997, pp. 807-823).

Hypertension and atherosclerosis or other hyperlipidemic conditions often coexist in a patient. It is possible that certain hyperlipidemic conditions such as atherosclerosis can have a direct or indirect affect on hypertension. For example, atherosclerosis frequently results in diminishment of the inside diameter of blood vessels. Furthermore, atherosclerosis frequently results in increased rigidity of blood vessels, including the aorta. Both diminished inside diameter of blood vessels and rigidity of blood vessels are factors which contribute to hypertension.

Myocardial infarction is the necrosis of heart muscle cells resulting from oxygen deprivation and is usually cause by an obstruction of the supply of blood to the affected tissue. For example, hyperlipidemia or hypercholesterolemia can cause the formation of atherosclerotic plaques, which can cause obstruction of blood flow and thereby cause myocardial infarction (E. Braunwald, Heart Disease, 5^{th} ed., W. B. Saunders & Co., Philadelphia, 1997, pp. 807-823). Another major risk factor for myocardial infarction is hypertension (E. Braunwald, Heart Disease, 5^{th} ed., W. B. Saunders & Co., Philadelphia, 1997, pp. 807-823). In other words, hypertension and hyperlipidemic conditions such as atherosclerosis or hypercholesterolemia work in concert to cause myocardial infarction.

Coronary heart disease is another disease, which is caused or aggravated by multiple factors including hyperlipidemic conditions and hypertension. Control of both hyperlipidemic conditions and hypertension are important to control symptoms or disease progression of coronary heart disease.

Angina pectoris is acute chest pain, which is caused by decreased blood supply to the heart. Decreased blood supply to the heart is known as myocardial ischemia. Angina pectoris can be the result of, for example, stenosis of the aorta, pulmonary stenosis and ventricular hypertrophy. Some antihypertensive agents, for example amlodipine, control angina pectoris by reducing peripheral resistance.

Some antihypertensive agents useful in the present invention are shown in Table 4, without limitation. A wide variety of chemical structures are useful as antihypertensive agents in the combinations of the present invention and the agents can operate by a variety of mechanisms. For example, useful antihypertensive agents can include, without limitation, an adrenergic blocker, a mixed alpha/beta adrenergic blocker, an alpha adrenergic blocker, a beta adrenergic blocker, an adrenergic stimulant, an angiotensin converting enzyme (ACE) inhibitor, an angiotensin II receptor antagonist, a calcium channel blocker, a diuretic, or a vasodilator. Additional hypertensive agents useful in the present invention are described by R. Scott in U.S. Patent Application No. 60/057,276 (priority document for PCT Patent Application No. WO 99/11260).

TABLE 4

| Antihypertensive | | | |
|---|---|---|---|
| Classification | Compound Name | Typical Dosage | |
| adrenergic blocker | Phenoxybenzamine | 1-250 | mg/day |
| adrenergic blocker | Guanadrel | 5-60 | mg/day |
| adrenergic blocker | Guanethidine | | |
| adrenergic blocker | Reserpine | | |
| adrenergic blocker | Terazosin | 0.1-60 | mg/day |
| adrenergic blocker | Prazosin | 0.5-75 | mg/day |
| adrenergic blocker | Polythiazide | 0.25-10 | mg/day |
| adrenergic stimulant | Methyldopa | 100-4000 | mg/day |
| adrenergic stimulant | Methyldopate | 100-4000 | mg/day |
| adrenergic stimulant | Clonidine | 0.1-2.5 | mg/day |
| adrenergic stimulant | Chlorthalidone | 10-50 | mg/day |
| adrenergic blocker | Guanfacine | 0.25-5 | mg/day |
| adrenergic stimulant | Guanabenz | 2-40 | mg/day |
| adrenergic stimulant | Trimethaphan | | |
| alpha/beta adrenergic blocker | Carvedilol | 6-25 | mg bid |
| alpha/beta adrenergic blocker | Labetalol | 10-500 | mg/day |
| beta adrenergic blocker | Propranolol | 10-1000 | mg/day |
| beta adrenergic blocker | Metoprolol | 10-500 | mg/day |
| alpha adrenergic blocker | Doxazosin | 1-16 | mg/day |
| alpha adrenergic blocker | Phentolamine | | |
| angiotensin converting enzyme | Quinapril | 1-250 | mg/day |
| inhibitor | | | |
| angiotensin converting enzyme | perindopril erbumine | 1-25 | mg/day |
| inhibitor | | | |
| angiotensin converting enzyme | Ramipril | 0.25-20 | mg/day |
| inhibitor | | | |
| angiotensin converting enzyme | Captopril | 6-50 | mg bid or tid |
| inhibitor | | | |
| angiotensin converting enzyme | Trandolapril | 0.25-25 | mg/day |
| inhibitor | | | |
| angiotensin converting enzyme | Fosinopril | 2-80 | mg/day |
| inhibitor | | | |
| angiotensin converting enzyme | Lisinopril | 1-80 | mg/day |
| inhibitor | | | |
| angiotensin converting enzyme | Moexipril | 1-100 | mg/day |
| inhibitor | | | |
| angiotensin converting enzyme | Enalapril | 2.5040 | mg/day |
| inhibitor | | | |
| angiotensin converting enzyme | Benazepril | 10-80 | mg/day |
| inhibitor | | | |
| angiotensin II receptor | candesartan cilexetil | 2-32 | mg/day |
| antagonist | | | |
| angiotensin II receptor | Inbesartan | | |
| antagonist | | | |
| angiotensin II receptor | Losartan | 10-100 | mg/day |
| antagonist | | | |
| angiotensin II receptor | Valsartan | 20-600 | mg/day |
| antagonist | | | |
| calcium channel blocker | Verapamil | 100-600 | mg/day |
| calcium channel blocker | Diltiazem | 150-500 | mg/day |
| calcium channel blocker | Nifedipine | 1-200 | mg/day |
| calcium channel blocker | Nimodipine | 5-500 | mg/day |
| calcium channel blocker | Delodipine | | |
| calcium channel blocker | Nicardipine | 1-20 | mg/hr i.v.; |
| | | 5-100 | mg/day oral |
| calcium channel blocker | Isradipine | | |
| calcium channel blocker | Amlodipine | 2-10 | mg/day |
| diuretic | Hydrochlorothiazide | 5-100 | mg/day |
| diuretic | Chlorothiazide | 250-2000 | mg bid or tid |
| diuretic | Furosemide | 5-1000 | mg/day |
| diuretic | Bumetanide | | |
| diuretic | ethacrynic acid | 20-400 | mg/day |
| diuretic | Amiloride | 1-20 | mg/day |
| Diuretic | Triameterene | | |
| Diuretic | Spironolactone | 5-1000 | mg/day |
| Diuretic | Eplerenone | 10-150 | mg/day |
| Vasodilator | Hydralazine | 5-300 | mg/day |
| Vasodilator | Minoxidil | 1-100 | mg/day |
| Vasodilator | Diazoxide | 1-3 | mg/kg |
| Vasodilator | Nitroprusside | | |

Additional calcium channel blockers which are useful in the combinations of the present invention include, without limitation, those shown in Table 5.

TABLE 5

| Compound Name | Reference |
|---|---|
| bepridil | U.S. Pat. No. 3,962,238 or |
| | U.S. Reissue No. 30,577 |
| clentiazem | U.S. Pat. No. 4,567,175 |
| diltiazem | U.S. Pat. No. 3,562,257 |
| fendiline | U.S. Pat. No. 3,262,977 |
| gallopamil | U.S. Pat. No. 3,261,859 |
| mibefradil | U.S. Pat. No. 4,808,605 |
| prenylamine | U.S. Pat. No. 3,152,173 |
| semotiadil | U.S. Pat. No. 4,786,635 |
| terodiline | U.S. Pat. No. 3,371,014 |
| verapamil | U.S. Pat. No. 3,261,859 |
| aranipine | U.S. Pat. No. 4,572,909 |
| bamidipine | U.S. Pat. No. 4,220,649 |
| benidipine | European Patent Application |
| | Publication No. 106,275 |
| cilnidipine | U.S. Pat. No. 4,672,068 |
| efonidipine | U.S. Pat. No. 4,885,284 |
| elgodipine | U.S. Pat. No. 4,962,592 |
| felodipine | U.S. Pat. No. 4,264,611 |
| isradipine | U.S. Pat. No. 4,466,972 |
| lacidipine | U.S. Pat. No. 4,801,599 |
| lercanidipine | U.S. Pat. No. 4,705,797 |
| manidipine | U.S. Pat. No. 4,892,875 |
| nicardipine | U.S. Pat. No. 3,985,758 |
| nifendipine | U.S. Pat. No. 3,485,847 |
| nilvadipine | U.S. Pat. No. 4,338,322 |
| nimodipine | U.S. Pat. No. 3,799,934 |
| nisoldipine | U.S. Pat. No. 4,154,839 |
| nitrendipine | U.S. Pat. No. 3,799,934 |
| cinnarizine | U.S. Pat. No. 2,882,271 |
| flunarizine | U.S. Pat. No. 3,773,939 |
| lidoflazine | U.S. Pat. No. 3,267,104 |
| lomerizine | U.S. Pat. No. 4,663,325 |
| Bencyclane | Hungarian Patent No. 151,865 |
| Etafenone | German Patent No. 1,265,758 |
| Perhexiline | British Patent No. 1,025,578 |

Additional ACE inhibitors which are useful in the combinations of the present invention include, without limitation, those shown in Table 6.

TABLE 6

| Compound Name | Reference |
|---|---|
| alacepril | U.S. Pat. No. 4,248,883 |
| benazepril | U.S. Pat. No. 4,410,520 |
| captopril | U.S. Pat. Nos. 4,046,889 and |
| | 4,105,776 |
| ceronapril | U.S. Pat. No. 4,452,790 |
| delapril | U.S. Pat. No. 4,385,051 |
| enalapril | U.S. Pat. No. 4,374,829 |
| fosinopril | U.S. Pat. No. 4,337,201 |
| imadapril | U.S. Pat. No. 4,508,727 |
| lisinopril | U.S. Pat. No. 4,555,502 |
| moveltopril | Belgian Patent No. 893,553 |
| perindopril | U.S. Pat. No. 4,508,729 |
| quinapril | U.S. Pat. No. 4,344,949 |
| ramipril | U.S. Pat. No. 4,587,258 |
| Spirapril | U.S. Pat. No. 4,470,972 |
| Temocapril | U.S. Pat. No. 4,699,905 |
| Trandolapril | U.S. Pat. No. 4,933,361 |

Additional beta adrenergic blockers which are useful in the combinations of the present invention include, without limitation, those shown in Table 7.

TABLE 7

| Compound Name | Reference |
|---|---|
| acebutolol | U.S. Pat. No. 3,857,952 |
| alprenolol | Netherlands Patent Application No. |
| | 6,605,692 |
| amosulalol | U.S. Pat. No. 4,217,305 |
| arotinolol | U.S. Pat. No. 3,932,400 |
| atenolol | U.S. Pat. No. 3,663,607 or |
| | U.S. Pat. No. 3,836,671 |
| befunolol | U.S. Pat. No. 3,853,923 |
| betaxolol | U.S. Pat. No. 4,252,984 |
| bevantolol | U.S. Pat. No. 3,857,981 |
| bisoprolol | U.S. Pat. No. 4,171,370 |
| bopindolol | U.S. Pat. No. 4,340,641 |
| bucumolol | U.S. Pat. No. 3,663,570 |
| bufetolol | U.S. Pat. No. 3,723,476 |
| bufuralol | U.S. Pat. No. 3,929,836 |
| bunitrolol | U.S. Pat. Nos. 3,940,489 and |
| | U.S. Pat. No. 3,961,071 |
| buprandolol | U.S. Pat. No. 3,309,406 |
| butiridine hydrochloride | French Patent No. 1,390,056 |
| butofilolol | U.S. Pat. No. 4,252,825 |
| carazolol | German Patent No. 2,240,599 |
| carteolol | U.S. Pat. No. 3,910,924 |
| carvedilol | U.S. Pat. No. 4,503,067 |
| celiprolol | U.S. Pat. No. 4,034,009 |
| cetamolol | U.S. Pat. No. 4,059,622 |
| cloranolol | German Patent No. 2,213,044 |
| dilevalol | Clifton et al., Journal of Medicinal |
| | Chemistry, 1982 25, 670 |
| epanolol | European Patent Publication |
| | Application No. 41,491 |
| indenolol | U.S. Pat. No. 4,045,482 |
| labetalol | U.S. Pat. No. 4,012,444 |
| levobunolol | U.S. Pat. No. 4,463,176 |
| mepindolol | Seeman et al., Helv. Chim. Acta, |
| | 1971, 54, 241 |
| metipranolol | Czechoslovakian Patent Application |
| | No. 128,471 |
| metoprolol | U.S. Pat. No. 3,873,600 |
| moprolol | U.S. Pat. No. 3,501,769 |
| nadolol | U.S. Pat. No. 3,935,267 |
| nadoxolol | U.S. Pat. No. 3,819,702 |
| nebivalol | U.S. Pat. No. 4,654,362 |
| nipradilol | U.S. Pat. No. 4,394,382 |
| oxprenolol | British Patent No. 1,077,603 |
| perbutolol | U.S. Pat. No. 3,551,493 |
| pindolol | Swiss Patent Nos. 469,002 and |
| | Swiss Patent Nos. 472,404 |
| practolol | U.S. Pat. No. 3,408,387 |
| pronethalol | British Pat. No. 909,357 |
| propranolol | U.S. Pat. Nos. 3,337,628 and |
| | U.S. Pat. Nos. 3,520,919 |
| sotalol | Uloth et al., Journal of Medicinal |
| | Chemistry, 1966, 9, 88 |
| sufinalol | German Pat. No. 2,728,641 |
| talindol | U.S. Pat. Nos. 3,935,259 and |
| | U.S. Pat. Nos. 4,038,313 |
| tertatolol | U.S. Pat. No. 3,960,891 |
| tilisolol | U.S. Pat. No. 4,129,565 |
| timolol | U.S. Pat. No. 3,655,663 |
| toliprolol | U.S. Pat. No. 3,432,545 |
| Xibenolol | U.S. Pat. No. 4,018,824 |

Additional alpha adrenergic blockers which are useful in the combinations of the present invention include, without limitation, those shown in Table 8.

TABLE 8

| Compound Name | Reference |
|---|---|
| amosulalol | U.S. Pat. No. 4,217,307 |
| arotinolol | U.S. Pat. No. 3,932,400 |
| dapiprazole | U.S. Pat. No. 4,252,721 |
| doxazosin | U.S. Pat. No. 4,188,390 |
| fenspirlde | U.S. Pat. No. 3,399,192 |
| indoramin | U.S. Pat. No. 3,527,761 |
| labetalol | U.S. Pat. No. 4,012,444 |
| naftopidil | U.S. Pat. No. 3,997,666 |
| nicergoline | U.S. Pat. No. 3,228,943 |
| prazosin | U.S. Pat. No. 3,511,836 |
| tamsulosin | U.S. Pat. No. 4,703,063 |
| Tolazoline | U.S. Pat. No. 2,161,938 |
| Trimazosin | U.S. Pat. No. 3,669,968 |
| Yohimbine | Raymond-Hamet, J. Pharm. Chim., |
| | 19, 209 (1934) |

Additional angiotensin II receptor antagonists, which are useful in the combinations of the present invention include, without limitation, those shown in Table 9.

TABLE 9

| Compound Name | Reference |
|---|---|
| Candesartan | U.S. Pat. No. 5,196,444 |
| Eprosartan | U.S. Pat. No. 5,185,351 |
| Irbesartan | U.S. Pat. No. 5,270,317 |
| Losartan | U.S. Pat. No. 5,138,069 |
| Valsartan | U.S. Pat. No. 5,399,578 |

Additional vasodilators which are useful in the combinations of the present invention include, without limitation, those shown in Table 10.

TABLE 10

| Compound Name | Reference |
|---|---|
| aluminum nicotinate | U.S. Pat. No. 2,970,082 |
| amotriphene | U.S. Pat. No. 3,010,965 |
| bamethan | Corrigan et al., Journal of the |
| | American Chemical Society, 1945, |
| | 67, 1894 |
| bencyclane | Hungarian Patent No. 151,865 |
| bendazol | J. Chem. Soc., 1968, 2426 |
| benfurodil hemisuccinate | U.S. Pat. No. 3,355,463 |
| benziodarone | U.S. Pat. No. 3,012,042 |
| betahistine | Walter et al., Journal of the American |
| | Chemical Society, 1941, 63, 2771 |
| bradykinin | Hamburg et al., Arch. Biochem. |
| | Biophys., 1958, 76, 252 |
| brovincamine | U.S. Pat. No. 4,146,643 |
| bufeniode | U.S. Pat. No. 3,542,870 |
| buflomedil | U.S. Pat. No. 3,895,030 |
| butalamine | U.S. Pat. No. 3,338,899 |
| cetiedil | French Patent No. 1,460,571 |
| chloracizine | British Patent No. 740,932 |
| chromonar | U.S. Pat. No. 3,282,938 |
| ciclonicate | German Patent No. 1,910,481 |
| cinepazide | Belgian Patent No. 730,345 |
| cinnarizine | U.S. Pat. No. 2,882,271 |
| citicoline | Kennedy et al., Journal of the |
| | American Chemical Society, 1955, |
| | 77,250 or synthesized as disclosed in |
| | Kennedy, Journal of Biological |
| | Chemistry, 1956, 222, 185 |
| clobenfural | British Patent No. 1,160,925 |
| clonitrate | see Annalen, 1870, 155, 165 |
| cloricromen | U.S. Pat. No. 4,452,811 |
| cyclandelate | U.S. Pat. No. 2,707,193 |
| diisopropylamine | Neutralization of dichloroacetic acid |
| dichloroacetate | with diisopropyl amine |
| diisopropylamine | British Patent No. 862,248 |
| dichloroacetate | |
| dilazep | U.S. Pat. No. 3,532,685 |
| dipyridamole | British Patent No. 807,826 |
| droprenilamine | German Patent No. 2,521,113 |
| ebumamonine | Hermann et al., Journal of the |
| | American Chemical Society, 1979, |
| | 101, 1540 |
| efloxate | British Patent Nos. 803,372 and |
| | 824,547 |
| eledoisin | British Patent No. 984,810 |
| erythrityl | May be prepared by nitration of |
| | erythritol according to methods well- |
| | known to those skilled in the art. See |
| | e.g., Merck Index. |
| etafenone | German Patent No. 1,265,758 |
| fasudil | U.S. Pat. No. 4,678,783 |
| fendiline | U.S. Pat. No. 3,262,977 |
| fenoxedil | U.S. Pat. No. 3,818,021 or German |
| | Patent No. 1,964,712 |
| floredil | German Patent No. 2,020,464 |
| flunarizine | German Patent No. 1,929,330 or |
| | French Patent No. 2,014,487 |
| flunarizine | U.S. Pat. No. 3,773,939 |
| ganglefene | U.S.S.R. Patent No. 115,905 |
| hepronicate | U.S. Pat. No. 3,384,642 |
| hexestrol | U.S. Pat. No. 2,357,985 |
| hexobendine | U.S. Pat. No. 3,267,103 |
| ibudilast | U.S. Pat. No. 3,850,941 |
| ifenprodil | U.S. Pat. No. 3,509,164 |
| iloprost | U.S. Pat. No. 4,692,464 |
| inositol | Badgett et al., Journal of the |
| | American Chemical Society, 1947, |
| | 69, 2907 |
| isoxsuprine | U.S. Pat. No. 3,056,836 |
| itramin tosylate | Swedish Patent No. 168,308 |
| kallidin | Biochem. Biophys. Re&Commun., |
| | 1961, 6, 210 |
| kallikrein | German Patent No. 1,102,973 |
| khellin | Baxter et al., Journal of the Chemical |
| | Society, 1949, S 30 |
| lidofiazine | U.S. Pat. No. 3,267,104 |
| lomerizine | U.S. Pat. No. 4,663,325 |
| mannitol hexanitrate | May be prepared by the nitration of |
| | mannitol according to methods well- |
| | known to those skilled in the art |
| medibazine | U.S. Pat. No. 3,119,826 |
| moxisylyte | German Patent No. 905,738 |
| nafronyl | U.S. Pat. No. 3,334,096 |
| nicametate | Blicke & Jenner, J. Am. Chem. Soc., |
| | 64, 1722 (1942) |
| nicergoline | U.S. Pat. No. 3,228,943 |
| nicofuranose | Swiss Patent No. 366,523 |
| nimodipine | U.S. Pat. No. 3,799,934 |
| nitroglycerin | Sobrero, Ann., 64, 398 (1847) |
| nylidrin | U.S. Pat. Nos. 2,661,372 and |
| | 2,661,373 |
| papaverine | Goldberg, Chem. Prod. Chem. News, |
| | 1954, 17, 371 |
| pentaerythritol tetranitrate | U.S. Pat. No. 2,370,437 |
| pentifylline | German Patent No. 860,217 |
| pentoxifylline | U.S. Pat. No. 3,422,107 |
| pentrinitrol | German Patent No. 638,422-3 |
| perhexilline | British Patent No. 1,025,578 |
| pimefylline | U.S. Pat. No. 3,350,400 |
| piribedil | U.S. Pat. No. 3,299,067 |
| prenylamine | U.S. Pat. No. 3,152,173 |
| propatyl nitrate | French Patent No. 1,103,113 |
| prostaglandin El | May be prepared by any of the |
| | methods referenced in the Merck |
| | Index, Twelfth Edition, Budaved, |
| | Ed., New Jersey, 1996, p. 1353 |
| suloctidil | German Patent No. 2,334,404 |
| tinofedrine | U.S. Pat. No. 3,563,997 |
| tolazoline | U.S. Pat. No. 2,161,938 |
| trapidil | East German Patent No. 55,956 |
| tricromyl | U.S. Pat. No. 2,769,015 |
| trimetazidine | U.S. Pat. No. 3,262,852 |
| trolnitrate phosphate | French Patent No. 984,523 or |
| | German Patent No. 830,955 |
| vincamine | U.S. Pat. No. 3,770,724 |
| vinpocetine | U.S. Pat. No. 4,035,750 |
| Viquidil | U.S. Pat. No. 2,500,444 |
| Visnadine | U.S. Pat. Nos. 2,816,118 and |
| | 2,980,699 |
| xanthinol niacinate | German Patent No. 1,102,750 or |
| | Korbonits et al., Acta. Pharm. Hung., |
| | 1968, 38, 98 |

Additional diuretics which are useful in the combinations of the present invention include, without limitation, those shown in Table 11.

TABLE 11

| Compound Name | Reference |
|---|---|
| Acetazolamide | U.S. Pat. No. 2,980,676 |
| Althiazide | British Patent No. 902,658 |
| Amanozine | Austrian Patent No. 168,063 |
| Ambuside | U.S. Pat. No. 3,188,329 |
| Amiloride | Belgian Patent No. 639,386 |
| Arbutin | Tschb&habln, Annalen, 1930, 479, |
| | 303 |
| Azosemide | U.S. Pat. No. 3,665,002 |
| Bendroflumethiazide | U.S. Pat. No. 3,265,573 |
| Benzthiazide | McManus et al., 136^{th} Am. Soc. |
| | Meeting (Atlantic City, September |
| | 1959). Abstract of Papers, pp 13-0 |
| benzylhydro- | U.S. Pat. No. 3,108,097 |
| chlorothiazide | |
| Bumetanide | U.S. Pat. No. 3,634,583 |
| Butazolamide | British Patent No. 769,757 |
| Buthiazide | British Patent Nos. 861,367 and |
| | 885,078 |
| Chloraminophenamide | U.S. Pat. Nos. 2,809,194, |
| | 2,965,655 and 2,965,656 |
| Chlorazanil | Austrian Patent No. 168,063 |
| Chlorothiazide | U.S. Pat. Nos. 2,809,194 and |
| | 2,937,169 |
| Chlorthalidone | U.S. Pat. No. 3,055,904 |
| Clofenamide | Olivier, Rec. Trav. Chim., 1918, 37, |
| | 307 |
| Clopamide | U.S. Pat. No. 3,459,756 |
| Clorexolone | U.S. Pat. No. 3,183,243 |
| Cyclopenthiazide | Belgian Patent No. 587,225 |
| Cyclothiazide | Whitehead et al., Journal of Organic |
| | Chemistry, 1961, 26, 2814 |
| Disulfamide | British Patent No. 851,287 |
| Epithiazide | U.S. Pat. No. 3,009,911 |
| ethacrynic acid | U.S. Pat. No. 3,255,241 |
| Ethiazide | British Patent No. 861,367 |
| Ethoxolamide | British Patent No. 795,174 |
| Etozolin | U.S. Pat. No. 3,072,653 |
| Fenquizone | U.S. Pat. No. 3,870,720 |
| Furosemide | U.S. Pat. No. 3,058,882 |
| Hydracarbazine | British Patent No. 856,409 |
| Hydrochlorothiazide | U.S. Pat. No. 3,164,588 |
| Hydroflumethiazide | U.S. Pat. No. 3,254,076 |
| Indapamide | U.S. Pat. No. 3,565,911 |
| Isosorbide | U.S. Pat. No. 3,160,641 |
| Mannitol | U.S. Pat. No. 2,642,462; or |
| | 2,749,371; or 2,759,024 |
| Mefruside | U.S. Pat. No. 3,356,692 |
| Methazolamide | U.S. Pat. No. 2,783,241 |
| Methyclothiazide | Close et al., Journal of the American |
| | Chemical Society, 1960, 82, 1132 |
| Meticrane | French Patent Nos. M2790 and |
| | 1,365,504 |
| Metochalcone | Freudenberg et al., Ber., 1957, 90, |
| | 957 |
| Metolazone | U.S. Pat. No. 3,360,518 |
| Muzolimine | U.S. Pat. No. 4,018,890 |
| Paraflutizide | Belgian Patent No. 620,829 |
| Perhexiline | British Patent No. 1,025,578 |
| Piretanide | U.S. Pat. No. 4,010,273 |
| Polythiazide | U.S. Pat. No. 3,009,911 |
| Quinethazone | U.S. Pat. No. 2,976,289 |
| Teclothiazide | Close et al., Journal of the American |
| | Chemical Society, 1960, 82, 1132 |
| Ticrynafen | U.S. Pat. No. 3,758,506 |
| Torasemide | U.S. Pat. No. 4,018,929 |
| Triamterene | U.S. Pat. No. 3,081,230 |
| Trichlormethiazide | deStevens et al., Experientia, 1960, |
| | 16, 113 |
| Tripamide | Japanese Patent No. 73 05,585 |
| Urea | Can be purchased from commercial |
| | sources |
| Xipamide | U.S. Pat. No. 3,567,777 |

VI. Treatment of Diseases

In one aspect of present invention, a method for treating cardiovascular disease in a host is provided by administering an effective amount of a compound of the following formula:

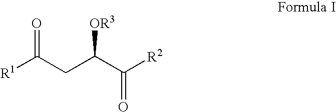

or a pharmaceutically acceptable salt, prodrug or active derivative thereof, wherein:

R¹ and R² are selected from the group consisting of OR⁴, alkyl, substituted alkyl, alkenyl, substituted alkenyl, alkynyi, substituted alkynyl, alkoxy, substituted alkyloxy, alkoxyalkyl, substituted alkoxyalkyl, NH₂, NHR⁵, NR⁷R⁶, mono- or polyhydroxy-substituted alkyl, aryl, substituted aryl, heteroaryl, substituted heteroaryl, acyloxy, substituted acyloxy, or haloalkyl, including CF₃; and,

R³ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, mono- or polyhydroxy-substituted alkyl, aryl, substituted aryl, heteroaryl, substituted heteroaryl, acyloxy, substituted acyloxy, alkylsulfonyl, arylsulfonyl, aralkylsulfonyl, amino acid residue, haloalkyl, including CF₃, or the carboxylic moiety of an ester, including CO-alkyl, CO-aryl, CO-alkoxyalkyl, CO-aryloxyalkyl, CO-substituted aryl; and,

R⁴ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy; and,

R⁵, R⁶, and R⁷ are selected from the group consisting of alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy.

In other embodiments of the present invention methods are provided for decreasing the serum lipoprotein cholesterol levels, decreasing the low density lipoprotein cholesterol levels, decreasing the very low density lipoprotein cholesterol levels, decreasing the serum triglyceride levels, decreasing the total serum cholesterol levels, and/or decreasing the serum triglyceride levels by administering an effective amount of a compound of Formula I (shown above).

In other aspects of the present invention methods are provided to treat and/or prevent the following diseases or conditions including, but not limited to: cardiovascular disease, hyperlipidemia, atherosclerosis, peripheral vascular disease, hypercholesterolemia, primary hyperlipidemia, secondary hyperlipidemia, hypothyroidism, chronic renal failure, nephrotic syndrome, cholestasis, familial combined hyperlipidaemia, familial hypercholesterolaemia, remnant hyperlipidaemia, chylo-micronaemia syndrome, familial hypertriglyceridaemia, obesitas, coronary atherosclerosis, ischaemic heart disease, cerebral vascular disease, acquired lipid disorders, acquired hyperlipoproteinemia; high blood cholesterol; high blood triglycerides; stroke, atherosclerosis, venous thrombosis, venous incompetence, vasculitis claudication, aneurysms, congestive heart failure, congenital heart disease, pericardial disease, valvular heart disease and/or cardiomyopathy, by administering an effective amount of a compound of Formula I (shown above).

The present invention now is described more fully by the following examples. This invention may, however, be embodied in many different forms and should not be construed as limited to the embodiments set forth herein; rather, these embodiments are provided so that this disclosure is thorough and complete, and fully conveys the scope of the invention to those skilled in the art.

### EXAMPLES

### Example 1

### The Effects of Malic Acid in the Genetically Obese Rat

The Zucker fa/fa rat model was selected to test the effects of malic acid supplements to the diet. The Zucker fa/fa rat is a genetically obese rat associated to elevated leptin levels in the blood. Among humans and most animal models, leptin is known to stimulate the desire to eat, leading to elevated caloric intake and obesity. Associated with the leptin production is an elevation in serum insulin (Giridharan 1998). Thus, obesity can arise from the increase food intake and/or the lipogenic effects of insulin on adipose tissue and liver.

Two separate experiments were conducted on the Zucker fa/fa rat. The first experiment served as a pilot study for the second. In Experiment I, ten male, 7-week-old Zucker fa/fa rats were divided into two parametric groups based upon the results of a clinical serum analysis for lipids, glucose, hepatic enzymes, and ions. At the age of 10 weeks, one group of five rats received 3 gm of D,L-malic acid, sodium salt (Sigma Chemical Co.)/liter of drinking tap water for twelve weeks. The control group of five rats received only tap water. After 2, 4, 7, and 10 and 12 weeks of treatment, 1-2 mL whole blood was collect from each rat via the tail caudal vein and the serum retained for analysis. All serum analysis was conducted in the out-patient clinical laboratory of the University Hospital of the University of Alabama at Birmingham with a Synchron LX System. The following methods for each parameter are used in their automated clinical systems.

TABLE 12
Zucker fa/fa Rat Serum Analytical Methods

| Serum Parameter | Analytical Procedures |
|---|---|
| Triglycerides | GPO method with Glycerol kinase, |
| | glycerophosphate oxidase @ 520 nm |
| Total | Cholesterolesterase, cholesterol esterase, |
| Cholesterol | peroxidase @ 520 nm |
| Cholesterol-HDL | Direct HDL with cholesterol esterase, oxidase |
| | @ 560 nm |
| Cholesterol-LDL | N-geneous LDL assay with cholesterol esterase |
| | and cholesterol oxidase @ 560 nm |
| Cholesterol-VLDL | Calculated |
| Glucose | Glucose oxidase with an oxygen electrode |
| AST | AST linked Malic acid Dehydrogenase @ 340 nm |
| ALT | ALT linked Lactate Dehydrogenase @ 340 nm |
| Chloride | Indirect potentiometry chloride electrode |
| | with Ag+ |
| Potassium | Indirect potentiometry with potassium selective |
| | electrode |
| Sodium | Indirect potentiometry with sodium selective |
| | electrodes |

In experiment II, forty male Zucker fa/fa rats at five to seven weeks of age began treatment similarly to the rats in Experiment I. At about 5 weeks of age, whole blood was collected from the tail caudal vein of each rat and lipid profiles were measured on the serum. At the age of about six weeks, rats were placed into the following four parametric groups of ten rats: Controls given drinking water; L-malic acid, sodium salt, 3 gm/liter drinking water; D,L-malic acid, sodium salt, 3 gm/liter drinking water; and D-malic acid, sodium salt, 3 gm/liter drinking water.

Food and water consumption was measured daily for each cage of two rats. Body weight was measured and tail caudal vein blood samples were collected at 6, 8, 10, 12, 14, 16, and 18 and 24 weeks of age. Rats received their oral treatment of malic acid isomers prior to the onset of hyperlipidemia and continued for 16 weeks. The lipid/liver profile was measured on the serum of each rate with the determination of serum triglycerides, total cholesterol, HDL, glucose, AST and ALT with the same procedures of Experiment I.

Results

Experiment I. This experiment served as a pilot study to test the hypolipidemic effects of D,L mixed isomers of malic acid. Table 13 lists the means±SEM of body weight and serum analysis from control and D,L-malic treated groups. The following were noted changes observed among the control group as an indication of the progression of the genetic disorder of the Zucker fa/fa rats. From 10 to 22 weeks of age, the Zucker fa/fa rat progressively increased in body weight at a rate of 32 grams/week. Furthermore, the hyperlipidemia of the Zucker fa/fa rat was first detected as early as 8 weeks of age when serum triglycerides were elevated to a mean of 389 mg %. Serum triglycerides significantly increased (P≦0.01) on biweekly bases in the Zucker fa/fa rats from week 10 to 20 weeks of age. At 20 weeks of age, the mean serum triglycerides for control Zucker fa/fa rats was 1,147 mg % with the higher values exceeding 2,600 mg %. Serum total cholesterol and HDL cholesterol levels were significantly increased (P≦0.05) on a monthly basis among control Zucker fa/fa rats. Serum AST and ALT significantly decreased (P≦0.05) from the 14^{th} week to the 18^{th} and 20^{th} week of age. Finially, the Zucker fa/fa rat did not exhibit any alteration in serum glucose, sodium or potassium, HCO₃, chloride, and BUN levels.

The results were similar for the D,L-malic acid treated Zucker fa/fa rat. D,L malic acid treatment had no effect on body weight of the Zucker fa/fa rat. However, D,L malic acid significantly decreased serum triglyceride levels (P≦0.01) from two to eight weeks of treatment. At ten and twelve weeks of treatment differences were more remarkable (P≦0.001). There was a significant decrease in serum total cholesterol (P≦0.05) after six weeks of treatment and serum HDL cholesterol (P≦0.05) after eight weeks of treatment. D,L malic acid significantly lowered serum AST and ALT (P≦0.05) after four and six weeks of treatment, but not beyond while having no detectable affect on serum glucose, sodium, or potassium, HCO₃, chloride, and BUN levels.

Table 14 lists the mean organ weights taken in control and D,L malic acid treated rats after 12 weeks of treatment (age 20 weeks). No significant differences were detected.

TABLE 14
Mean ± SEM Body Weight and Serum Parameters of Control and D,L
Malic Acid Orally Treated Zucker fa/fa Rats.

| | | −2 weeks | 0 weeks | 2 weeks | 4 weeks | 7 weeks | 10 weeks | 12 weeks |
|---|---|---|---|---|---|---|---|---|
| Parameter | Treatment | Treatment | Treatment | Treatment | Treatment | Treatment | Treatment | Treatment |
| Body Wt. | Control | 363 ± 5 | 496 ± 7 | 580 ± 21 | 610 ± 17 | 687 ± 20 | 722 ± 20 | 747 ± 21 |
| | Malic | 359 ± 6 | 484 ± 10 | 553 ± 14 | 592 ± 15 | 660 ± 19 | 708 ± 26 | 722 ± 30 |
| Serum | Control | 391 ± 31 | — | 645 ± 104** | 829 ± 102** | 721 ± 75** | 668 ± 70*** | 1147 ± 272* |
| Triglyceride | Malic | 387 ± 41 | — | 358 ± 51 | 522 ± 82 | 422 ± 44 | 430 ± 39 | 437 ± 35 |
| Serum T | Control | 85 ± 3 | — | 109 ± 10 | 114 ± 12 | 150 ± 15* | 156 ± 19* | 210 ± 38* |
| Chol | Malic | 91 ± 4 | — | 96 ± 5 | 99 ± 5 | 120 ± 8 | 123 ± 14 | 137 ± 14 |
| Serum HDL | Control | 86 ± 2 | — | 97 ± 7 | 92 ± 6 | 120 ± 5 | 131 ± 14* | 160 ± 19** |
| | Malic | 90 ± 3 | — | 92 ± 4 | 87 ± 3 | 110 ± 5 | 107 ± 10 | 117 ± 9 |
| Serum LDL | Control | — | — | — | — | — | 6.8 ± 4.7 | 17 ± 8 |
| | Malic | — | — | — | — | — | 1.4 ± 1.4 | 6 ± 0.4 |
| Serum | Control | — | — | — | — | — | 22 ± 18 | 29 ± 11 |
| VLDL | Malic | — | — | — | — | — | 4 ± 4 | 14 ± 5 |
| Serum AST | Control | — | — | — | 202 ± 28* | 193 ± 26** | 88 ± 7 | 115 ± 14 |
| | Malic | — | — | — | 145 ± 13 | 126 ± 6 | 94 ± 2 | 115 ± 16 |
| Serum ALT | Control | — | — | — | 108 ± 16* | 115 ± 17** | 67 ± 4 | 68 ± 5 |
| | Malic | — | — | — | 64 ± 4 | 68 ± 2 | 69 ± 4 | 71 ± 6 |
| Serum | Control | 155 ± 8 | — | 159 ± 17 | 155 ± 12 | 186 ± 31 | 143 ± 11 | 137 ± 12 |
| Glucose | Malic | 147 ± 2 | — | 167 ± 14 | 156 ± 12 | 173 ± 10 | 141 ± 10 | 153 ± 9 |
| Serum Na+ | Control | 146 ± 0.7 | — | 141 ± 0.4 | 143 ± 2.0 | 142 ± 0.7 | 141 ± 0.2 | 141 ± 0.6 |
| | Malic | 147 ± 0.2 | — | 141 ± 0.6 | 143 ± 1.1 | 144 ± 0.4 | 143 ± 0.8 | 141 ± 0.6 |
| Serum K+ | Control | 5.8 ± 0.7 | — | 6.3 ± 0.1 | 6.0 ± 0.1 | 6.0 ± 0.1 | 6.9 ± 0.3 | 7.0 ± 0.2 |
| | Malic | 6.1 ± 0.2 | — | 5.6 ± 0.3 | 5.8 ± 0.1 | 5.4 ± 0.2 | 6.6 ± 0.2 | 6.4 ± 0.1 |
| Serum Cl− | Control | 99 ± 0.9 | — | 92 ± 1.5 | 94 ± 1.2 | 93 ± 1.8 | 96 ± 0.7 | 94 ± 1.0 |
| | Malic | 101 ± 0.7 | — | 95 ± 1.7 | 98 ± 0.9 | 96 ± 0.7 | 98 ± 0.9 | 98 ± 0.7 |
| Serum BUN | Control | 21 ± 2.0 | — | 12 ± 0.7 | 12 ± 1.4 | 12 ± 0.6 | 17 ± 0.9 | 16 ± 0.5 |
| | Malic | 21 ± 0.9 | — | 13 ± 0.5 | 12 ± 0.6 | 11 ± 0.3 | 16 ± 1.2 | 16 ± 5 = 0.9 |
| Serum | Control | 0.48 ± 0.03 | — | .38 ± .02 | .34 ± .02 | .30 ± 0 | .32 ± .02 | .32 ± .02 |
| Creatine | Malic | 0.46 ± 0.02 | — | .42 ± .02 | .38 ± .02 | .34 ± .02 | .32 ± .02 | .34 ± .02 |

*P ± .05;
**P ± .01;
***P ± .001

TABLE 15
Mean Organ Weights (gram ± SEM) of Control and
12 week D, L Malic Acid Treated Zucker fa/fa Rats

| | | | | Epididymal |
|---|---|---|---|---|
| Treatment | Liver | Heart | Left Kidney | Fat |
| Control | 32.44 ± 2.39 | 1.590 ± .062 | 2.434 ± .253 | 0.445 ± .042 |
| D, L Malic | 27.69 ± 1.91 | 1.564 ± .050 | 1.914 ± .087 | 0.350 ± .050 |
| Acid | | | | |

Histological examination of the aorta of the 12 week (20 weeks of age) control and D,L malic acid treated Zucker fa/fa rats reveal no pathology associated with plaque formation that might be an early sign of atherosclerosis. The medial lobe of the livers from these same rats revealed a moderately advanced stage of fat deposits. From liver sections cut at 8 um thickness, 200 hepatocytes were quantified for the percent of fatty inclusions from 0 to 100%. There were no significant differences between control and D,L malic acid treated rats.

TABLE 16
Mean and Range of Percent Fatty Deposition in Hepatocytes of
Control and 12 week treated D, L Malic Acid Zucker fa/fa Rats

| | Control | D,L Malic Treated |
|---|---|---|
| Mean of 200 cells/rat ± SEM | 47.94 ± 1.57 | 45.69 ± 0.62 |
| Range of cellular fat deposits | 10% to 95% | 8% to 90% |

Tables 17 and 18 list tissue levels of purine nucleotides and dinucleotides, respectively, in frozen-acid extracted liver from Control and D,L malic acid treated rats after 12 weeks treatment. No significant differences in mean hepatic purine nucleotides between control and treated rats were detected. The energy status of hepatocytes based upon the ratio of high to low energy nucleotides was greater in D,L malic acid treated rats, but the means were not significantly different.

TABLE 17
Mean Hepatic Purine Nucleotides Level (nmoles/gm tissue wet
wt. ± SEM) in Control and D,L Malic Acid Treated Zucker fa/fa Rats.

| Treatment | Adenosine | AMP | ADP | ATP | AT/DMado* |
|---|---|---|---|---|---|
| Control | 78 ± 22 | 194 ± 15 | 134 ± 9 | 591 ± 73 | 1.49 ± .22 |
| D,L Malic | 65 | 168 ± 18 | 125 ± 7 | 556 ± 35 | 1.61 ± .18 |

*AT/DNado = ratio of tissue levels of ATP/ADP + AMP + Adenosine

Hepatic tissue levels of the purine dinucleotides were especially interesting in that the mean of NAD levels were elevated but not significantly. However, the tissue levels of NADP were significantly elevated in D,L malic acid treated rats.

TABLE 18
Mean Hepatic Purine Dinucleotide Levels (nmoles/gm
tissue wet wt. ± SEM) in Control and D, L,
Malic Acid Treated Zucker fa/fa Rats.

| Treatment | NAD | NADP |
|---|---|---|
| Control | 335 ± 15 | 42.8 ± 16.7 |
| D,L-Malic Acid | 350 ± 23 | 61.7 ± 3.8* |

*Significantly different P ≦ 0.05.

Biopsies for Pathology and Nucleotide Analysis

After eight weeks of treatment, Experiment I was concluded with all ten rats being anesthetized with pentobarbital (40 mg/kg, i.p). Immediately prior to the rat's euthanasia, a portion of the medial lobe of the liver was weighed, immediately frozen, pulverized and extracted in 12% perchloric acid in dry ice for nucleotide analysis. The acid extracted tissue was thawed, neutralized in saturated potassium bicarbonate and centrifuged at 10,000×g for 15 minutes. The final supernatant was analyzed for nucleotides by HPLC according to the method of Jenkins, et al (1988). In addition to body weight, the heart, liver, epididymal fat and left kidney were weighed. The aorta and a portion of the medial lobe of the liver were fixed in buffered 10% formalin and prepared for paraffin embedding and sectioning for pathological analysis.

Experiment II. This follow-up experiment doubled the number of rats per group (ten/group) and included treatment groups to distinguish the roles of the D isomer and the L isomer of malic acid (Control, L-Malic, D,L-Malic, and D-Malic Acid).

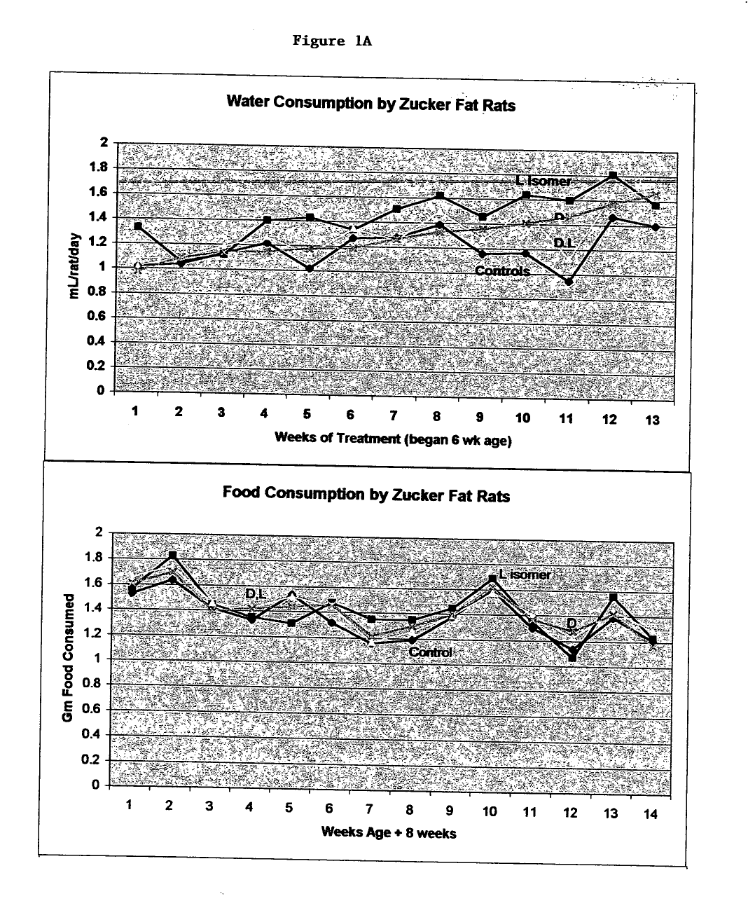

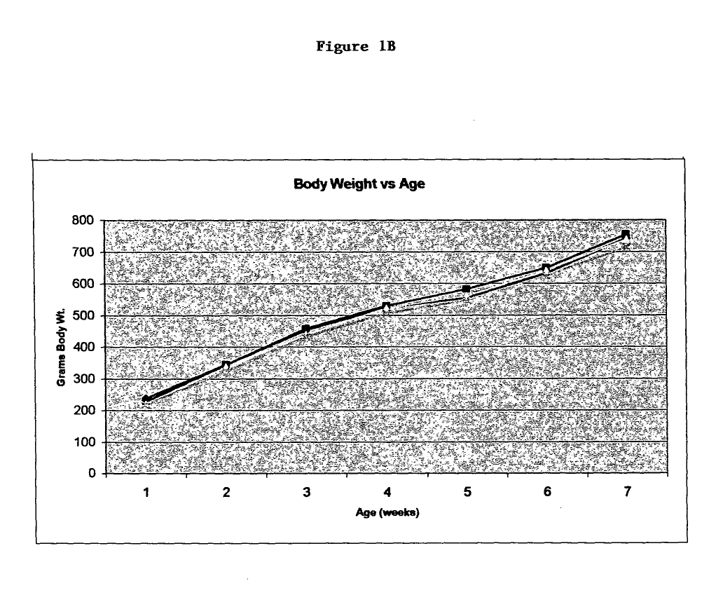

FIG. 1 indicates the following about food and water consumption of the Zucker fa/fa rat: Control Zucker fa/fa rats did not significantly alter the rate of food consumption from 6 to 20 weeks of age, averaging 1.40 grams of rat chow consumed per rat per day. While Zucker fa/fa rats maintained on L-malic acid tended to eat more rat chow on a daily bases, no statistical significance was detected between any group of rats on the mean weight of rat chow consumed/rat/day. All groups significantly increased mean body weight on a biweekly basis. No statistical significant difference between mean body weights were detected between controls and treated groups at any age. The mean volume of water consumed by each group increased on a monthly basis throughout the experiment. Significant differences of means between groups for water consumption occurred only between the L-malic acid treated group and control at 10 and 11 weeks of treatment. D-malic acid and D,L malic acid groups did not differ from the L-malic acid or control groups. Measuring the consumption of water among the D and D,L malic acid treated rats allowed for the determination of dosage. The consumption of water in the D and D,L groups increased from 1.0 mL/rat/day to 1.6 mL/rat/day throughout the 24 weeks of treatment. At an administration rate of 3gm malic acid/L of drinking water, these rats began consumption at the first week of treatment of 3 mg malic acid/rat/day. After 24 weeks of treatment, these same rats were consuming 4.8 mg malic acid/rat/day.

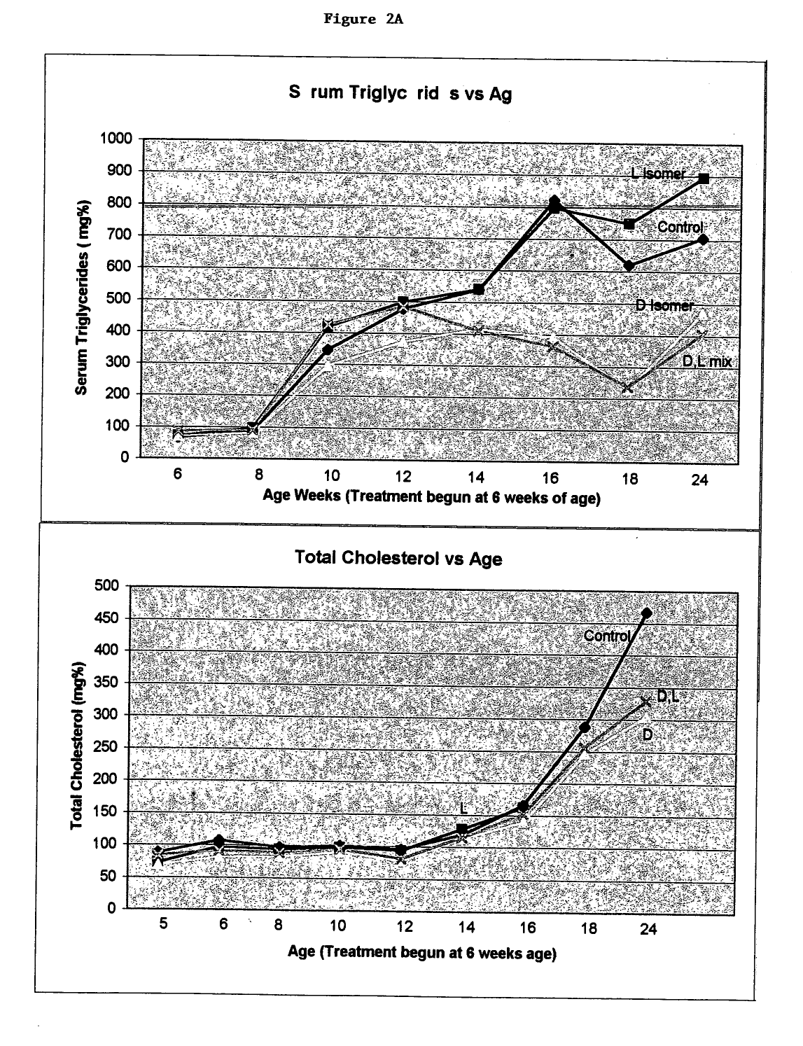

FIG. 2 illustrates the following about the affects of the isomers of malic acid on serum lipid profiles. Among control and L-malic acid treated Zucker fa/fa rats mean serum triglycerides increased significantly from 8 to 24 weeks of age on a biweekly basis. There was no significant difference between the mean serum levels of triglycerides, total cholesterol between control and L-malic acid treated Zucker fa/fa rats. After 24 weeks of age (18 weeks of treatment) the rats treated with L-malic acid had greater mean serum triglycerides than controls but not statistically significant. After 12 weeks and beyond of D-malic acid treatment Zucker fa/fa rats had significantly (P≦0.05) lowered mean serum triglycerides compared to controls. After 18 and 24 weeks of treatment both D malic acid and D,L malic acid groups mean serum triglycerides were significantly (P≦0.01) decreased below controls. Mean serum total cholesterol was significantly (P≦0.05) decreased below controls after 18 and 24 weeks of treatment. No significant differences in means serum HDL cholesterol was noted between the Zucker fa/fa rat groups. Furthermore, there were no significant difference between the four groups in mean serum AST and ALT levels. There were no difference in means serum glucose between groups, except that in L-malic acid treated Zucker fa/fa rats was sporadically (only weeks 8 and 16) elevated (P≦0.05) above control Zucker fa/fa rats.

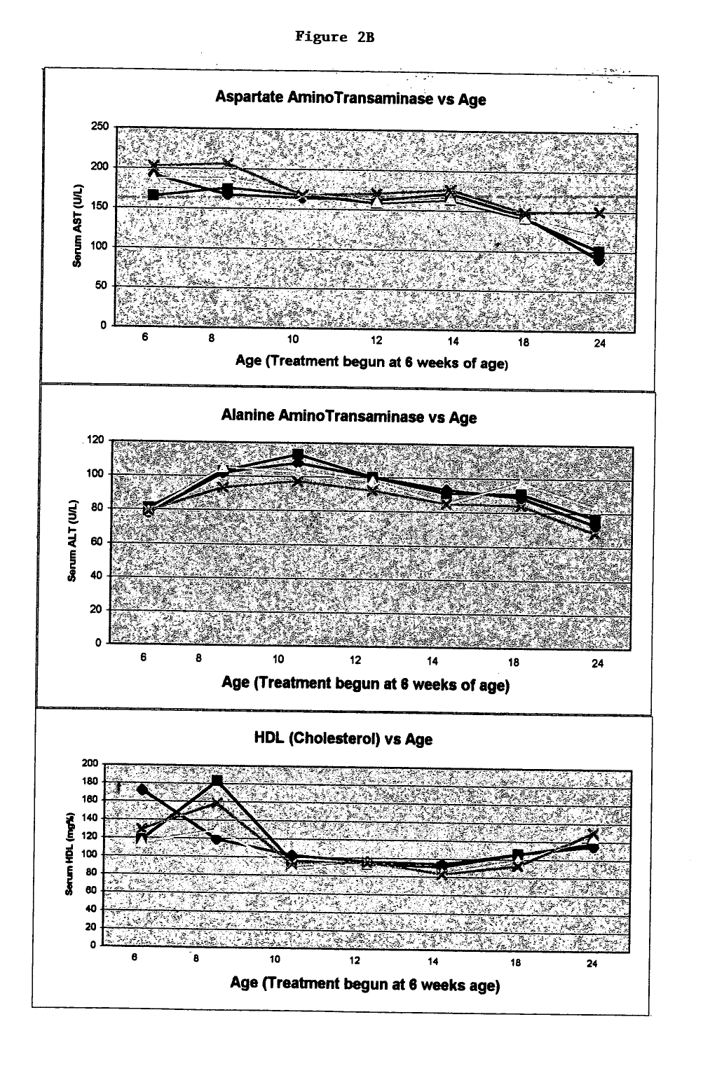

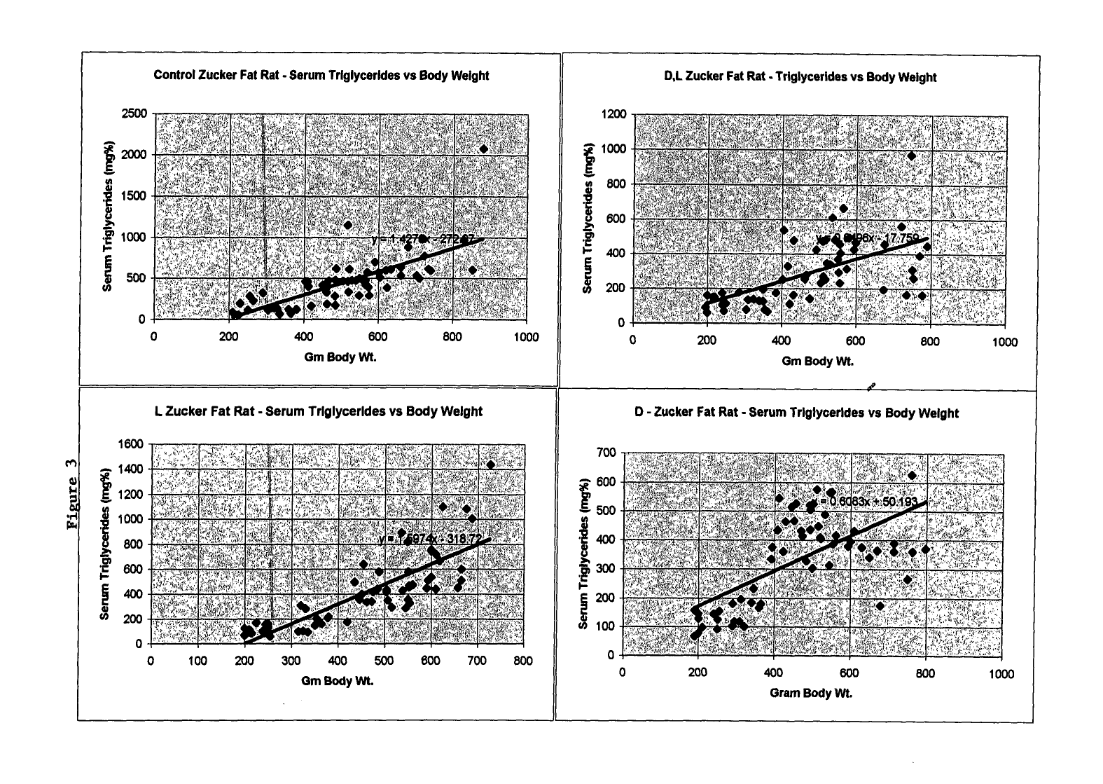

In Experiment II, it was difficult to obtain forty Zucker fa/fa rats of the same litter age. Subsequently, the rats used in this experiment ranged in age by 12 days. Additionally, the data for serum triglycerides proved to be more strongly correlated to body weight rather than to duration of treatment. In FIG. 3 values for serum triglycerides were plotted relative to body weight for the four groups through the sixteen weeks of treatment. The slope of the linear regressions indicated that the controls and the D treated rats were essentially the same and significantly different (P≦0.01) from the controls.

### Example 2

### Investigation into the Possible Mechanism of Action of D-Malic Acid

To investigate the mechanism of action of D-malic acid as a hypolipidemic agent, we concluded Experiment II after 16 weeks of treatment with D-malic acid by an analysis of electrophoretic isoenzymes of hepatic malic enzyme, decarboxylating (1.1.1.40). One aged male Sprague-Dawley rat, three control Zucker fa/fa rats and four DO-malic acid treated Zucker fa/fa rats were give an anesthetic dose of pentobarbital. From the living rat, two to three grams of the medial lobe of the liver was excised, weighed, minced, and homogenized in cold (4° C.) sucrose buffer (250 mM sucrose, 50 mM Tris, pH 7.2) in a volume five time the gram weight of the tissue. Cell debri and nuclei were removed by centrifugation (600×g at 4° C. From this supernatant, mitochondria were removed at 10,000×g at 4° C. Isozymes of malic enzyme were electrophoretically separated and stained from the 10,000×g supernatant according to the method of Harris and Hopkins (1976).

Results

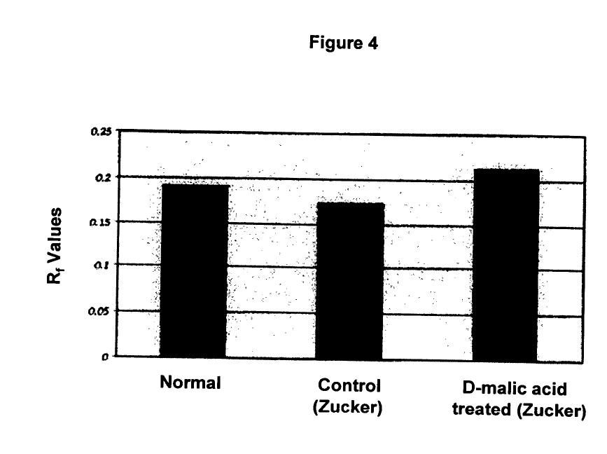

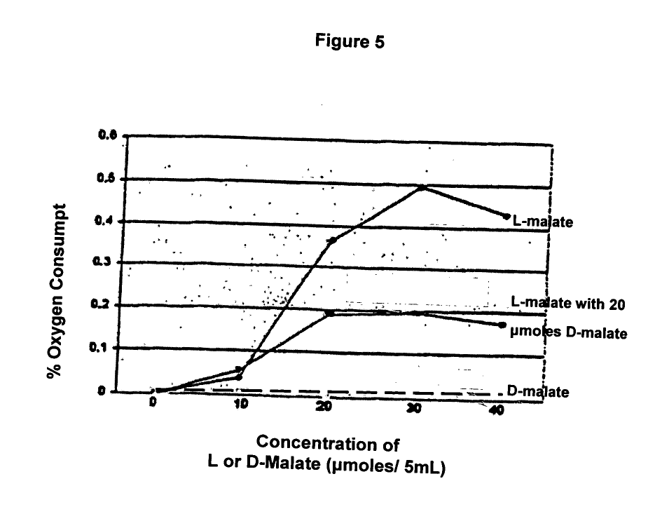

Evidence for a Mechanism of D-Malic Acid. At the conclusion of Experiment II described above under Example 1, electorphoretic isozymes of hepatic malic enzyme was demonstrated in one 4-month old, male Sprague Dawley, two Control Zucker fa/fa rats and three D-malic acid treated Zucker fa/fa rats. FIG. 4 illustrates that the electrophoretic anodal migration of malic enzyme in the hyperlipidemic Zucker fa/fa rats is less than normolipidemic Sprague Dawley rat. Furthermore, after 24 weeks of treatment with D-malic acid, the Zucker fa/fa isozyme of hepatic malic enzyme was similar to the Sprague-Dawley rat than the hyperlipidemic model. FIG. 4 depicts the electrophoretic isoenzymes of cytosolic malic enzyme, decarboxylating (1.1.1.40) illustrating the anodal Rf values. FIG. 5 shows the percent oxygen consumption of mitochondria from a normal Sprague-Dawley rat and demonstrates that mitochondria from a normal Sprague-Dawley rat can metabolize L-malic acid with the consumption of oxygen. The metabolism of L-malic acid saturates at 30 umole/5 mL (or 6 mM). Additionally, D-malic acid is not metabolized at concentrations less than 40 umoles/5 mL (6.7 mM). More important, 20 umoles/5 mL D-malic acid inhibited the metabolism of L-malic acid by mitochondria.

Results

Oral malic acid has a significant hypolipidemic effect in the genetic Zucker obese rat. Above all, the therapeutics of this compound is isomer dependent. The L-isomer of malic acid, which is the form used by cellular enzymes and machinery has no effect on serum lipid levels. It is the D-isomer that is effective; and this isomer is not usable as an energy source by the cellular machinery.

D-malic acid was administered orally in drinking water at 3 gm/L. Early in the study, 8 week-old rats consumed an average of 13.6 mg D-malic acid/kg body wt./day. At 20 weeks of age, these same rats consumed 7.23 mg D-malic acid/kg body wt/day. Furthermore, the D,L malic acid treated rats were consuming roughly half of the active ingredient and still they exhibited a significant hypolipidemic effect. While this study does not attempt to determine effective or threshold dosages, it is evident in Zucker fa/fa rats that dosages between 13.6 mg/kg/day and 3.6 mg/kg/day are effective in lowering serum lipids.

While the Zucker rat or human cell can not utilize D-malic acid, this compound does occur naturally. Ligand exchange liquid chromatography has been used to separate and measure the D and L isomers from fruit (Benecke, 1984). Apple juice contains approximately 600 mg/100 mL of malic acid with the L (96.7%) in far excess of the D (3.3%) isomer. Eisele (1996) measured D-malic acid in juice from Brix apples which ranged from 26 to 188 mg/100 mL.

Although not bound by any discussion of mechanism of action of the hypolipidemic effect of D-malic acid, the following suggests that D-malic acid could inhibit short chain fatty acid synthesis in adipose tissue and liver. This, in turn, would lead to reduced serum triglyceride. D-malic acid is not metabolized by rat liver mitochondria. If it is to have an effect at the cellular level it must be extra-mitochondrial. D-malic acid is capable of blocking the metabolism of L-malic acid, which is transported into the mitochondria, enters the citric acid cycle and generates energy in the form of NADH and citrate. NADH can be used in electron transport to product ATP. Citrate once transported into the cytoplasm is the precursor for fatty acid synthesis. Malic enzyme, carboxylating (1.1.1.40) is a cytoplasmic enzyme necessary in fatty acid synthesis. First, malic enzyme is involved in the shuttling of L-malic acid back into the mitochondria. Second, malic enzyme generates NADPH, which is necessary in the later dehydrogenase steps of fatty acid synthesis. In D-malic enzyme treated rats the hepatic malic enzyme is eletrophoretically altered after 20 weeks of treatment. After 12 weeks of D,L malic acid treatment, the levels of liver NADP is elevated. The primary relationship between malic acid and NADP is through malic enzyme.

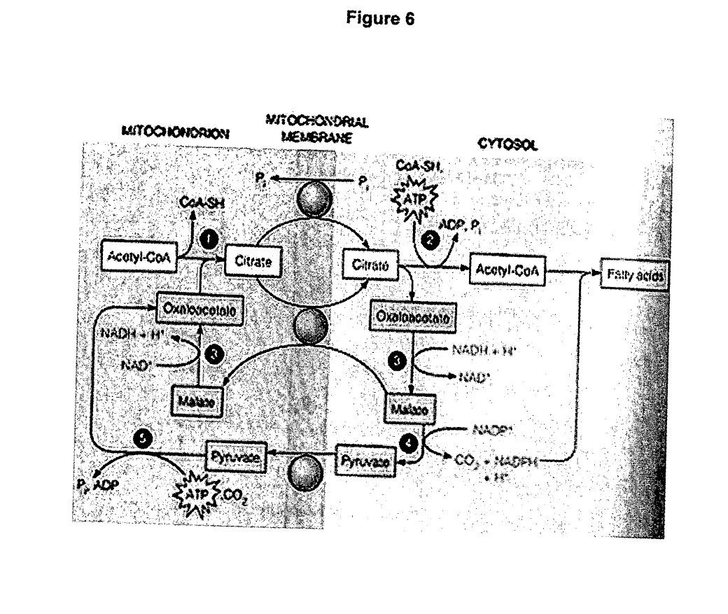

The following is one possible hypolipidemic mechanism for the hypolipideic effects of D-malic acid. D-malic acid binds to and inhibits cytosolic malic enzyme, decarboxylating (1.1.1.40). This reduces the necessary production of NADPH for fatty acid synthesis as well as the conversion of malic acid to pyruvate. Without the reuptake of pyruvate into the mitochondria, the conversion to citrate is reduced as is the cytosolic production of acetyl CoA (see Step 4, FIG. 6).

Mitochondrial incubations indicated that D-malic acid partially blocks the transport of L-malic acid into the mitrochondria. This also explains why a D,L isomer mixture of malic acid works as well as D-malic acid, alone. Blocking the transport of L-malic acid into the mitochondria would reduce the production of citrate in the mitrochondria and subsequently the synthesis of fatty acid.

### Example 3

### The Effect of D-Malic Acid on Hepatic Mitochondria

To determine the effect of D-malic acid on hepatic mitochondria, mitochondria from the Sprague Dawley rat used for isoenzyme studies were prepared according to the method of Johnson and Lardy (1967). The 60033 g pellet was resuspended in cold sucrose buffer and recentrifuged at 600×g. This supernatant was mixed with the original 600×g supernatant. The 10,000×g mitochondria pellet was suspended in 5 mL of sucrose buffer. Mitochondrial oxygen uptake was measured in response to L-malic acid, D-malic acid and combinations of D and L malic acid with the same incubation buffer of Blair (1967) in a YSI Biological Oxygen Monitor, model 5300. Consumption of oxygen was represented as percent of oxygen saturation.

The means of all parametric data was compared with ANOVA when the means of multiple groups were compared. A t-test was used to compare the difference in means of two groups. Statistical significance was accepted at the 5 percent level (P≦0.05) (Steel and Torrie, 1960).

Many modifications and other embodiments of the invention come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

We claim:

1. A method for treating cardiovascular disease in a host comprising administering an effective amount of a compound of the following formula: 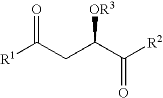
or a pharmaceutically acceptable salt, prodrug or active derivative thereof, wherein:
R¹ and R² are selected from the group consisting of OR⁴, alkyl, substituted alkyl, alkenyl, substituted alkenyl, alkynyi, substituted alkynyl, alkoxy, substituted alkyloxy, alkoxyalkyl, substituted alkoxyalkyl, NH₂, NHR⁵, NR⁷R⁶, mono- or polyhydroxy-substituted alkyl, aryl, substituted aryl, heteroaryl, substituted heteroaryl, acyloxy, substituted acyloxy, or haloalkyl; and,
R³ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, mono- or polyhydroxy-substituted alkyl, aryl, substituted aryl, heteroaryl, substituted heteroaryl, acyloxy, substituted acyloxy, alkylsulfonyl, arylsulfonyl, aralkylsulfonyl, amino acid residue, haloalkyl, or the carboxylic moiety of an ester; and,
R⁴ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy; and,
R⁵, R⁶, and R⁷ are selected from the group consisting of alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy.

2. The method of claim 1, wherein the compound is in substantially pure form.

3. The method of claim 1, wherein the compound is D-malic acid.

4. The method of claim 1, wherein the compound is D,L-malic acid.

5. A method for decreasing the serum lipoprotein cholesterol level in a host comprising administering an effective amount of a compound of the following formula: 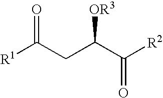
or a pharmaceutically acceptable salt, prodrug or active derivative thereof, wherein:
R¹ and R² are selected from the group consisting of OR⁴, alkyl, substituted alkyl, alkenyl, substituted alkenyl, alkynyl, substituted alkynyl, alkoxy, substituted alkyloxy, alkoxyalkyl, substituted alkoxyalkyl, NH₂, NHR⁵, NR⁷R⁶, mono- or polyhydroxy-substituted alkyl aryl, substituted aryl, heteroaryl, substituted heteroaryl, acyloxy, substituted acyloxy, or haloalkyl; and,
R³ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, mono- or polyhydroxy-substituted alkyl, aryl, substituted aryl, heteroaryl, substituted heteroaryl, acyloxy, substituted acyloxy, alkylsulfonyl, arylsulfonyl, aralkylsulfonyl, amino acid residue, haloalkyl, or the carboxylic moiety of an ester; and,
R⁴ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy; and,
R⁵, R⁶, and R⁷ are selected from the group consisting of alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy.

6. The method of claim 5, wherein the compound is in substantially pure form.

7. The method of claim 5, wherein the compound is D-malic acid.

8. The method of claim 5, wherein the compound is D,L-malic acid.

9. A method for decreasing the low density lipoprotein cholesterol level in a host comprising administering an effective amount of a compound of the following formula: 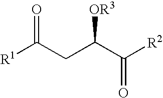
or a pharmaceutically acceptable salt, prodrug or active derivative thereof, wherein:
R¹ and R² are selected from the group consisting of OR⁴, alkyl, substituted alkyl, alkenyl, substituted alkenyl, alkynyl, substituted alkynyl, alkoxy, substituted alkyloxy, alkoxyalkyl, substituted alkoxyalkyl, NH₂, NHR⁵, NR⁷R⁶, mono- or polyhydroxy-substituted alkyl, aryl, substituted aryl, heteroaryl, substituted heteroaryl, acyloxy, substituted acyloxy, or haloalkyl; and,
R³ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, mono- or polyhydroxy-substituted alkyl, aryl, substituted aryl, heteroaryl, substituted heteroaryl, acyloxy, substituted acyloxy, alkylsulfonyl, arylsulfonyl, aralkylsulfonyl, amino acid residue, haloalkyl, or the carboxylic moiety of an ester; and,
R⁴ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy; and,
R⁵, R⁶, and R⁷ are selected from the group consisting of alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy.

10. The method of claim 9, wherein the compound is in substantially pure form.

11. The method of claim 9, wherein the compound is D-malic acid.

12. The method of claim 9, wherein the compound is D,L-malie acid.

13. A method for decreasing the very low density lipoprotein cholesterol level in a host comprising administering an effective amount of a compound of the following formula: 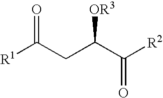
or a pharmaceutically acceptable salt, prodrug or active derivative thereof, wherein:
R¹ and R² are selected from the group consisting of OR⁴, alkyl, substituted alkyl, alkenyl, substituted alkenyl, alkynyl, substituted alkynyl, alkoxy, substituted alkyloxy, alkoxyalkyl, substituted alkoxyalkyl, NH₂, NHR⁵, NR⁷R⁶, mono- or polyhydroxy-substituted alkyl, aryl, substituted aryl, heteroaryl, substituted heteroaryl, acyloxy, substituted acyloxy, or haloalkyl; and,
R³ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, mono- or polyhydroxy-substituted alkyl, aryl, substituted aryl, heteroaryl, substituted heteroaryl, acyloxy, substituted acyloxy, alkylsulfonyl, arylsulfonyl, aralkylsulfonyl, amino acid residue, haloalkyl, or the carboxylic moiety of an ester; and,
R⁴ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy; and,
R⁵, R⁶, and R⁷ are selected from the group consisting of alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy.

14. The method of claim 13, wherein the compound is in substantially pure form.

15. The method of claim 13, wherein the compound is D-malic acid.

16. The method of claim 13, wherein the compound is D,L-malic acid.

17. A method for decreasing the serum triglyceride level in a host comprising administering an effective amount of a compound of the following formula: 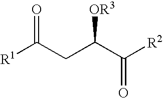
or a pharmaceutically acceptable salt, prodrug or active derivative thereof, wherein:
R¹ and R² are selected from the group consisting of OR⁴, alkyl, substituted alkyl, alkenyl, substituted alkenyl, alkynyl, substituted alkynyl, alkoxy, substituted alkyloxy, alkoxyalkyl, substituted alkoxyalkyl, NH₂, NHR⁵, NR⁷R⁶, mono- or polyhydroxy-substituted alkyl, aryl, substituted aryl, heteroaryl, substituted heteroaryl, acyloxy, substituted acyloxy, or haloalkyl; and,
R³ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, mono- or polyhydroxy-substituted alkyl, aryl, substituted aryl, heteroaryl, substituted heteroaryl, acyloxy, substituted acyloxy, alkylsulfonyl, arylsulfonyl, aralkylsulfonyl, amino acid residue, haloalkyl, or the carboxylic moiety of an ester; and,
R⁴ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy; and,
R⁵, R⁶, and R⁷ are selected from the group consisting of alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy.

18. The method of claim 17, wherein the compound is in substantially pure form.

19. The method of claim 17, wherein the compound is D-malic acid.

20. The method of claim 18, wherein the compound is D,L-malic acid.

21. A method for decreasing the total serum cholesterol level in a host comprising administering an effective amount of a compound of the following formula: 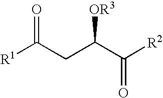
or a pharmaceutically acceptable salt, prodrug or active derivative thereof, wherein:
R¹ and R² are selected from the group consisting of OR⁴, alkyl, substituted alkyl, alkenyl, substituted alkenyl, alkynyl, substituted alkynyl, alkoxy, substituted alkyloxy, alkoxyalkyl, substituted alkoxyalkyl, NH₂, NHR⁵, NR⁷R⁶, mono- or polyhydroxy-substituted alkyl, aryl, substituted aryl, heteroaryl, substituted heteroaryl, aeyloxy, substituted acyloxy, or haloalkyl; and,
R³ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, mono- or polyhydroxy-substituted alkyl, aryl, substituted aryl, heteroaryl, substituted heteroaryl, acyloxy, substituted acyloxy, alkylsulfonyl, arylsulfonyl, aralkylsulfonyl, amino acid residue, haloalkyl, or the carboxylic moiety of an ester; and,
R⁴ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy; and,
R⁵, R⁶, and R⁷ are selected from the group consisting of alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy.

22. The method of claim 21, wherein the compound is in substantially pure form.

23. The method of claim 21, wherein the compound is D-malic acid.

24. The method of claim 21, wherein the compound is D,L-malic acid.

25. The method of claim 1, further comprising administering a compound in combination or alternation selected from the group consisting of statins, IBAT inhibitors, MTP inhibitors, cholesterol absorption antagonists, phytosterols, CETP inhibitors, fibric acid derivatives and antihypertensive agents.

26. The method of claim 25, further comprising the administration of the compound (−)-(2R,4S)-4-Amino-2-2-ethyl-6-trifluoromethyl-3,4-dihydro-2H-quinoline-1-carboxylic acid ethyl ester or its salts.

27. The method of claim 25, wherein the fibric acid derivative is selected from the group consisting of clofibrate, fenofibrate, ciprofibrate, bezafibrate and gemfibrozil.

28. A pharmaceutical composition for decreasing the serum lipoprotein cholesterol level in a host consisting essentially of a compound of the following formula: 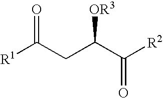
or a pharmaceutically acceptable salt, prodrug or active derivative thereof, wherein:
R¹ and R² are selected from the group consisting of OR⁴, alkyl, substituted alkyl, alkenyl, substituted alkenyl, alkynyl, substituted alkynyl, alkoxy, substituted alkyloxy, alkoxyalkyl, substituted alkoxyalkyl, NH₂, NHR⁵, NR⁷R⁶, mono- or polyhydroxy-substitUted alkyl, aryl, substituted aryl, heteroaryl, substituted heteroaryl, acyloxy, substituted acyloxy, or haloalkyl; and,
R³ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, mono- or polyhydroxy-substituted alkyl, aryl, substituted aryl, heteroaryl, substituted heteroaryl, acyloxy, substituted acyloxy, alkylsulfonyl, arylsulfonyl, aralkylsulfonyl, amino acid residue, haloalkyl, or the carboxylic moiety of an ester; and,
R⁴ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy; and,
R⁵, R⁶, and R⁷ are selected from the group consisting of alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy.

29. The pharmaceutical composition of claim 28, wherein the compound is in substantially pure form.

30. The pharmaceutical composition of claim 28, wherein the compound is D-malic acid.

31. The pharmaceutical composition of claim 28, wherein the compound is D,L-malic acid.

32. The pharmaceutical composition of claim 28, wherein the serum lipoprotein is selected from LDL, VLDL and HDL.

33. A pharmaceutical composition for decreasing the serum total cholesterol level in a host consisting essentially of a compound of the following formula: 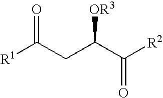
or a pharmaceutically acceptable salt, prodrug or active derivative thereof, wherein:
R¹ and R² are selected from the group consisting of OR⁴, alkyl, substituted alkyl, alkenyl, substituted alkenyl, alkynyl, substituted alkynyl, alkoxy, substituted alkyloxy, alkoxyalkyl, substituted alkoxyalkyl, NH₂, NHR⁵, NR⁷R⁶, mono- or polyhydroxy-substituted alkyl, aryl, substituted aryl, heteroaryl, substituted heteroaryl, acyloxy, substituted acyloxy, or haloalkyl; and,
R³ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, mono- or polyhydroxy-substituted alkyl, aryl, substituted aryl, heteroaryl, substituted heteroaryl, acyloxy, substituted acyloxy, alkylsulfonyl, arylsulfonyl, aralkylsulfonyl, amino acid residue, haloalkyl, or the carboxylic moiety of an ester; and,
R⁴ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy; and,
R⁵, R⁶, and R⁷ are selected from the group consisting of alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy.

34. The pharmaceutical composition of claim 33, wherein the compound is in substantially pure form.

35. The pharmaceutical composition of claim 33, wherein the compound is D-malic acid.

36. The pharmaceutical composition of claim 33, wherein the compound is D,L-malic acid.

37. A pharmaceutical composition for decreasing the serum triglyceride level in a host consisting essentially of a compound of the following formula: 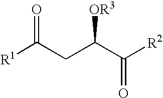
or a pharmaceutically acceptable salt, prodrug or active derivative thereof, wherein:
R¹ and R² are selected from the group consisting of OR⁴, alkyl, substituted alkyl, alkenyl, substituted alkenyl, alkynyl, substituted alkynyl, alkoxy, substituted alkyloxy, alkoxyalkyl, substituted alkoxyalkyl, NH₂, NHR⁵, NR⁷R⁶, mono- or polyhydroxy-substituted alkyl, aryl, substituted aryl, heteroaryl, substituted heteroaryl, acyloxy, substituted acyloxy, or haloalkyl; and,
R³ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, mono- or polyhydroxy-substituted alkyl, aryl, substituted aryl, heteroaryl, substituted heteroaryl, acyloxy, substituted acyloxy, alkylsulfonyl, arylsulfonyl, aralkylsulfonyl, amino acid residue, haloalkyl, or the carboxylic moiety of an ester; and,
R⁴ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy; and,
R⁵, R⁶, and R⁷ are selected from the group consisting of alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy.

38. The pharmaceutical composition of claim 37, wherein the compound is in substantially pure form.

39. The pharmaceutical composition of claim 37, wherein the compound is D-malic acid.

40. The pharmaceutical composition of claim 37, wherein the compound is D,L-malic acid.

41. The pharmaceutical composition of claim 28, further comprising a compound selected from the group consisting of statins, JBAT inhibitors, MTP inhibitors, cholesterol absorption antagonists, phytosterols, CETP inhibitors, fibric acid derivatives and antihypertensive agents.

42. The pharmaceutical composition of claim 41, further comprising the compound (−)-(2R,4S)-4-Amino-2-2-ethyl-6-trifluoromethyl-3,4-dihydro-2H-quinoline-1-carboxylic acid ethyl ester or its salts.

43. The composition of claim 41, wherein the fibric acid derivative is selected from the group consisting of clofibrate, fenofibrate, ciprofibrate, bezafibrate and gemfibrozil.

44. A method for treating hyperlipidemia comprising administering to a host an effective amount of a compound of the following formula: 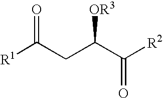
or a pharmaceutically acceptable salt, prodrug or active derivative thereof, wherein:
R¹ and R² are selected from the group consisting of OR⁴, alkyl, substituted alkyl, alkenyl, substituted alkenyl, alkynyl, substituted alkynyl, alkoxy, substituted alkyloxy, alkoxyalkyl, substituted alkoxyalkyl, NH₂, NHR⁵, NR⁷R⁶, mono- or polyhydroxy-substituted alkyl, aryl, substituted aryl, heteroaryl, substituted heteroaryl, acyloxy, substituted acyloxy, or haloalkyl; and,
R³ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, mono- or polyhydroxy-substituted alkyl, aryl, substituted aryl, heteroaryl, substituted heteroaryl, acyloxy, substituted acyloxy, alkylsulfonyl, arylsulfonyl, aralkylsulfonyl, amino acid residue, haloalkyl, or the carboxylic moiety of an ester; and,
R⁴ is selected from the group consisting of hydrogen, alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy; and,
R⁵, R⁶, and R⁷ are selected from the group consisting of alkyl, substituted alkyl, alkenyl, substituted alkenyl, alkynyl, substituted alkynyl, alkoxy, substituted alkyloxy, alkoxyalkyl, substituted alkoxyalkyl, substituted aryl, heteroaryl, substituted heteroaryl, acyloxy, or substituted acyloxy.

45. The method of claim 44, wherein the compound is in substantially pure form.

46. The pharmaceutical composition of claim 44, wherein the compound is D-malic acid.

47. The pharmaceutical composition of claim 44, wherein the compound is D,L-malic acid.
